(19) United States
(12) Patent Application Publication
(10) Pub. No.: US 2010/0305140 A1
(43) Pub. Date: Dec. 2, 2010

(54) BENZIMIDAZOLE DERIVATIVES WHICH ARE TO BE USED AS ANTAGONIST FOR THE CB1-RECEPTOR

(75) Inventors: William Brown, Saint Laurent (CA); Daniel Page, Saint Laurent (CA); Sanjay Srivastava, Saint Laurent (CA); Christopher Walpole, Saint Laurent (CA); Zhong-Yong Wei, Saint Laurent (CA); Hua Yang, Verdun (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/303,643

(22) PCT Filed: Jun. 11, 2007
(86) PCT No.: PCT/SE2007/000562

§ 371 Date: Aug. 18, 2010

Related U.S. Application Data
(60) Provisional application No. 60/804,599, filed on Jun. 13, 2006.

(51) Int. Cl. A61K 31/496; C07D 403/10; C07D 401/12; A61K 31/4184; A61K 31/454; A61P 1/00

(52) U.S. Cl. 514/254.06; 548/306.1; 546/199; 544/370; 514/394; 514/322

(57) ABSTRACT

Compounds of formula I or pharmaceutically acceptable salts thereof: wherein X, A, R¹, R², R³ and R⁴ are as defined in the specification as well as salts and pharmaceutical compositions including the compounds are prepared. They are useful in therapy, in particular in the management of pain.

### BACKGROUND OF THE INVENTION

1. Field of the Invention

The invention is related to therapeutic compounds, pharmaceutical compositions containing these compounds, manufacturing processes thereof and uses thereof. Particularly, the present invention is related to compounds that may be effective in treating pain, cancer, multiple sclerosis, Parkinson's disease, Huntington's chorea, Alzheimer's disease, anxiety disorders, gastrointestinal disorders and/or cardiovascular disorders.

2. Discussion of Relevant Technology

Pain management has been an important field of study for many years. It has been well known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located is predominately in the central nervous system, whereas CB₂ receptors are located primarily in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side effects, e.g., psychoactive side effects, the abuse potential, drug dependence and tolerance, etc. These undesired side effects are known to be mediated by the CB₁ receptors located in CNS. There are lines of evidence, however, suggesting that CB1 agonists acting at peripheral sites or with limited CNS exposure can manage pain in humans or animals with much improved overall in vivo profile.

Therefore, there is a need for new CB₁ receptor ligands such as agonists that may be useful in managing pain or treating other related symptoms or diseases with reduced or minimal undesirable CNS side effects.

### DESCRIPTION OF THE EMBODIMENTS

The present invention provides CB₁ receptor ligands which may be useful in treating pain and/or other related symptoms or diseases.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “alkynyl” used alone or as suffix or prefix, refers to a monovalent straight or branched chain hydrocarbon radical having at least one carbon-carbon triple bond and comprising at least 2 up to about 12 carbon atoms. The triple bond of an alkynyl group can be unconjugated or conjugated to another unsaturated group. Suitable alkynyl groups include, but are not limited to, C₂₋₆alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl can be unsubstituted or substituted with one or two suitable substituents.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to link two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “heteroaryl” used alone or as a suffix or prefix, refers to a heterocyclyl having aromatic character.

The term “heterocylcoalkyl” used alone or as a suffix or prefix, refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” refers to —NH₂.

Halogen includes fluorine, chlorine, bromine and iodine.

“Halogenated,” used as a prefix of a group, means one or more hydrogens on the group are replaced with one or more halogens.

“RT”, “r.t.” or “rt” means room temperature.

“DMF” refers to dimethyl formamide.

“DIPEA” refers to N,N-diisopropylethylamine.

“HATU” refers to 2-(7-Aza-1H-benzotriazole-1-yl)-1,1,3,3-tetramethyluronium hexafluorophosphate.

One aspect of the invention is a compound of formula I, a pharmaceutically acceptable salt thereof, a diastereomer, an enantiomer, or a mixture thereof:

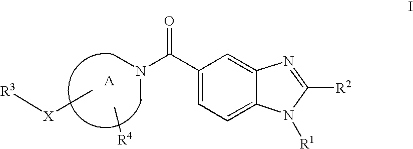

wherein:

R¹ is selected from C₁₋₁₀alkyl, C₂₋₁₀alkenyl, C₁₋₁₀alkoxy, C₆₋₁₀aryl-C₁₋₆alkyl, C₆₋₁₀aryl-C(═O)-C₁₋₆alkyl, C₃₋₁₀cycloalkyl-C₁₋₆alkyl, C₄₋₈cycloalkenyl-C₁₋₆alkyl, C₃₋₆heterocyclyl-C₁₋₆alkyl, C₃₋₆heterocyclyl-C(═O)—C₁₋₆alkyl, C₆₋₁₀aryl, C₆₋₁₀aryl-C(═O)—, C₃₋₁₀cycloalkyl, C₄₋₈cycloalkenyl, C₃₋₆heterocyclyl and C₃₋₆heterocyclyl-C(═O)—; wherein said C₁₋₁₀alkyl, C₂₋₁₀alkenyl, C₁₋₁₀alkoxy, C₆₋₁₀aryl-C₁₋₆alkyl, C₆₋₁₀aryl-C(═O)—C₁₋₆alkyl, C₃₋₁₀cycloalkyl-C₁₋₆alkyl, C₄₋₈cycloalkenyl-C₁₋₆alkyl, C₃₋₆heterocyclyl-C₁₋₆alkyl, C₃₋₆heterocyclyl-C(═O)—C₁₋₆alkyl, C₆₋₁₀aryl, C₆₋₁₀aryl-C(═O)—, C₃₋₁₀cycloalkyl, C₄₋₈cycloalkenyl, C₃₋₆heterocyclyl or C₃₋₆heterocyclyl-C(═O)— is optionally substituted by one or more groups selected from carboxy, —(C═O)—NH₂, halogen, cyano, nitro, methoxy, ethoxy, methyl, ethyl, hydroxy, —N(R⁶)—C(═O)R⁵, —S(═O)₂—NR⁵R⁶, —C(═O)—NR⁵R⁶, —NH—C(═O)—NR⁵R⁶ and —NR⁵R⁶;

R² is selected from the group consisting of C₁₋₁₀alkyl, C₂₋₁₀alkenyl, C₂₋₁₀alkynyl, C₃₋₈cycloalkyl, C₃₋₈cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl and C₃₋₆heterocycloalkyl, wherein said C₁₋₁₀alkyl, C₂₋₁₀alkenyl, C₂₋₁₀alkynyl, C₃₋₈cycloalkyl, C₃₋₈cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl or C₃₋₆heterocycloalkyl used in defining R² is optionally substituted by one or more groups selected from carboxy, —(C═O)—NH₂, halogen, cyano, nitro, methoxy, ethoxy, methyl, ethyl, hydroxy, and —NR⁵R⁶;

R³ is selected from C₁₋₆alkyl, C₂₋₆alkenyl, C₃₋₆cycloalkyl, C₃₋₆cycloalkyl-C₁₋₄alkyl, C₂₋₅heteroaryl, C₂₋₅heteroaryl-C₁₋₄alkyl, C₂₋₅heterocycloalkyl, C₂₋₆heterocycloalkyl-C₁₋₄alkyl, phenyl and benzyl, wherein said C₁₋₆alkyl, C₂₋₆alkenyl, C₃₋₆cycloalkyl, C₃₋₆cycloalkyl-C₁₋₄alkyl, C₂₋₅heteroaryl, C₂₋₅heteroaryl-C₁₋₄alkyl, C₂₋₅heterocycloalkyl, C₂₋₅heterocycloalkyl-C₁₋₅alkyl, phenyl or benzyl is optionally substituted by one or more groups selected from C₁₋₆alkyl, carboxy, halogen, cyano, nitro, methoxy, ethoxy, hydroxy, and —NR⁵R⁶; and

R⁴ is selected from C₁₋₆alkyl, carboxy, halogen, cyano, nitro, methoxy, ethoxy, hydroxy, and —NR⁵R⁶;

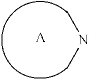

is a 4, 5 or 6-membered heterocycle which optionally contains one or two additional heteroatoms selected from O, S and N on its ring in addition to the nitrogen shown;

X is selected from —O—C(═O)—, —C(═O)—NH—, —NH—C(═O)—, —NHR⁷—C(═O)—, —C(═O)—NHCH₂—, —NH—C(═O)CH₂—, —NH—C(═O)—NH—, —O—C(═O)—NH—, —NH—(CH₂)ₘ—, —O—(CH₂)ₘ—, —C(═O)—O—, and —NH—C(═O)—O—;

wherein R⁵ and R⁶ are independently selected from —H, C₁₋₆alkyl optionally substituted with —OH, methoxy, ethoxy or halogen, C₃₋₆cycloalkyl-C₀₋ₘalkyl optionally substituted with —OH, methoxy, ethoxy or halogen, C₂₋₆alkenyl optionally substituted with —OH, methoxy, ethoxy or halogen, and a divalent C₁₋₆alkylene optionally substituted with —OH, methoxy, ethoxy or halogen that together with another divalent R⁵ or R⁶ form a portion of a ring;

R⁷ is C₁₋₆alkyl, and

m is 0, 1, 2 or 3.

In a particular embodiment, R¹ is selected from C₃₋₇cycloalkyl-C₁₋₂alkyl and C₂₋₆heterocycloalkyl-C₁₋₂alkyl, wherein said C₃₋₇cycloalkyl or C₂₋₆heterocycloalkyl is optionally substituted with one or more groups selected from carboxy, —C(═O)—NH₂, halogen, cyano, nitro, methoxy, ethoxy, methyl, ethyl, hydroxy, and amino.

In another particular embodiment, R¹ is selected from cyclohexylmethyl and tetrahydropyranylmethyl wherein said cyclohexylmethyl or tetrahydropyranylmethyl is optionally substituted with one or more groups selected from carboxy, —C(═O)—NH₂, halogen, cyano, nitro, methoxy, ethoxy, methyl, ethyl, hydroxy, and amino.

In a further embodiment, R¹ is selected from cyclohexylmethyl and tetrahydropyranylmethyl wherein said cyclohexylmethyl or tetrahydropyranylmethyl is optionally substituted with one or more groups selected from methyl, hydroxy, chloro, fluoro and bromo.

In an even further embodiment, R¹ is selected from cyclohexylmethyl and tetrahydropyran-4-ylmethyl wherein said cyclohexylmethyl or tetrahydropyran-4-ylmethyl is optionally substituted with one or more groups selected from chloro and fluoro.

In a yet further embodiment, R¹ is selected from cyclohexylmethyl, (4,4-difluorocyclohexyl)methyl, (4-fluorocyclohexyl)methyl and tetrahydro-2H-pyran-4-ylmethyl.

In another particular embodiment, R² is selected from C₁₋₆alkyl, C₂₋₆alkenyl, C₃₋₆cycloalkyl, and C₃₋₆cycloalkyl-C₁₋₂alkyl, wherein said C₁₋₆alkyl, C₂₋₆alkenyl, C₃₋₆cycloalkyl, or C₃₋₆cycloalkyl-C₁₋₂alkyl is optionally substituted by one or more groups selected from halogen, methoxy, ethoxy, methyl, ethyl, and hydroxy.

In a further embodiment, R² is selected from propyl, isopropyl, n-butyl, isobutyl, t-butyl, 1-pentyl, 2-pentyl, 3-pentyl, 1,1-dimethyl-1-propyl, 3-methyl-1-butyl, 1,1-difluoroethyl and 2,2-dimethyl-1-propyl, wherein said propyl, isopropyl, n-butyl, isobutyl, t-butyl, 1-pentyl, 2-pentyl, 3-pentyl, 1,1-dimethyl-1-propyl, 3-methyl-1-butyl, or 2,2 dimethyl-1-propyl is optionally substituted by one or more groups selected from halogen, methoxy and ethoxy.

In an even further embodiment, R² is selected from propyl, isopropyl, n-butyl, isobutyl, t-butyl, 1-pentyl, 2-pentyl, 3-pentyl, 1,1-dimethyl-1-propyl, 3-methyl-1-butyl, 1,1-difluoroethyl and 2,2-dimethyl-1-propyl.

In an even further embodiment, R² is selected from t-butyl, 1,1-difluoroethyl and 1,1-dimethyl-1-propyl.

In a particular embodiment, R³ is selected from hydrogen, C₁₋₄alkyl, halogenated C₁₋₄alkyl, hydroxy-C₁₋₄alkyl, C₃₋₆cycloalkyl, C₃₋₆cycloalkyl-C₁₋₂alkyl, methoxy-C₁₋₄alkyl, ethoxy-C₁₋₄alkyl, and C₂₋₄alkenyl.

Particularly, R⁴ is selected from hydrogen, hydroxy, halogen, isocyanato, methoxy, ethoxy, C₁₋₄alkyl, halogenated C₁₋₄alkyl, phenyl, benzyl, amino, C₃₋₆cycloalkyl, C₃₋₆cycloalkyl-C₁₋₂alkyl, and C₁₋₄alkoxymethyl.

Particularly,

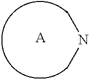

is selected from piperidinyl, isoxazolindinyl, azetidinyl, morpholinyl, pyrazolyl, pyrrolyl and pyrrolidinyl.

In a particular embodiment, R⁴ is hydrogen.

In a particular embodiment, X is selected from —O—C(═O)—, —C(O)—NH—, —NH—C(═O)—, —C(═O)—NHCH₂—, —NH—C(═O)CH₂—, —NH—C(═O)—NH—, —O—C(═O)—NH—, —NH—, —O—, —C(═O)—O—, and —NH—C(═O)—O—.

In another particular embodiment, —X—R³ is selected from cyclobutanylcarbonylamino, hydrocarbonyl, 2-hydroxyethylaminocarbonyl, isopropylaminocarbonyl, cyclobutanylaminocarbonyl, ethylaminocarbonyl, cyclopropylaminocarbonyl, methoxycarbonyl, ethoxycarbonyl, t-butoxycarbonyl, t-butoxycarbonylamino, allylaminocarbonyl, methylaminocarbonyl, aminocarbonyl, 2-fluoroethylaminocarbonyl, propylaminocarbonyl, cyclopropylmethylaminocarbonyl, cyclobutylmethylaminocarbonyl, t-butoxycarbonylamino, ethylaminocarbonylamino, isocyanato, cyclopropylaminocarbonylamino, 2-hydroxyethylaminocarbonylamino, ethylaminocarboxy, acetylamino, propionylamino, ethylaminocarbonylmethyl, 2-fluoroethylaminocarbonylmethyl, 2,2-difluoroethylaminocarbonyl, 2,2-difluoroethylaminocarbonylmethyl, acetylaminomethyl, cyclopropylcarbonylaminomethyl, propionylaminomethyl, and methylaminocarbonylmethyl.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation or chiral resolution of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

It will also be understood that certain compounds of the present invention may exist in solvated, for example hydrated, as well as unsolvated forms. It will further be understood that the present invention encompasses all such solvated forms of the compounds of the formula I.

Within the scope of the invention are also salts of the compounds of the formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB₁ receptors. More particularly, the compounds of the invention exhibit activity as agonist of the CB₁ receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of CB₁ receptors is present or implicated. Furthermore, the compounds of the invention may be used to treat cancer, multiple sclerosis, Parkinson's disease, Huntington's chorea, Alzheimer's disease, anxiety disorders, gastrointestinal disorders and cardiavascular disorders.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of cannabinoid receptors is present or implicated in that paradigm. This may involve the use of isotopically labeled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung edema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anesthetic state (e.g. amnesia, analgesia, muscle relaxation and sedation). Included in this combination are inhaled anesthetics, hypnotics, anxiolytics, neuromuscular blockers and opioids.

Another aspect of the present invention is the use of a compound according to Formula I, for the inhibition of transient lower esophageal sphincter relaxations (TLESRs) and thus for treatment or prevention of gastroesophageal reflux disorder (GERD). The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. In yet further embodiments of the present invention, the compound according to Formula I are useful for the prevention of reflux, treatment or prevention of regurgitation, treatment or prevention of asthma, treatment or prevention of laryngitis, treatment or prevention of lung disease and for the management of failure to thrive.

A further aspect of the present invention is the use of a compound according to Formula I, for the manufacture of a medicament for the inhibition of transient lower esophageal sphincter relaxations, for the treatment or prevention of GERD, for the prevention of reflux, for the treatment or prevention of regurgitation, treatment or prevention of asthma, treatment or prevention of laryngitis, treatment or prevention of lung disease and for the management of failure to thrive.

Still another aspect of the present invention is the use of a compound according to Formula I for the manufacture of a medicament for the treatment or prevention of functional gastrointestinal disorders, such as functional dyspepsia (FD). Yet another aspect of the present invention is the use of a compound according to Formula I for the manufacture of a medicament for the treatment or prevention of irritable bowel syndrome (IBS), such as constipation predominant IBS, diarrhea predominant IBS or alternating bowel movement predominant IBS. Exemplary irritable bowel syndrome (IBS) and functional gastrointestinal disorders (FGD), such as functional dyspepsia (FD), are illustrated in Thompson W G, Longstreth G F, Drossman D A, Heaton K W, Irvine E J, Mueller-Lissner S A. C. Functional Bowel Disorders and Functional Abdominal Pain. In: Drossman D A, Talky N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000: 351-432 and Drossman D A, Corazziari E, Talky N J, Thompson W G and Whitehead W E. Rome II: A multinational consensus document on Functional Gastrointestinal Disorders. Gut 45(Suppl.2), II1-II81.9-1-1999.

Also within the scope of the present invention is the use of any of the compounds according to the Formula I above, for the manufacture of a medicament for the treatment of any of the conditions discussed above.

A further aspect of the invention is a method for the treatment of a subject suffering from any of the conditions discussed above, whereby an effective amount of a compound is according to the formula I above, is administered to a patient in need of such treatment.

Thus, the invention provides a compound of formula I, or pharmaceutically acceptable salt or solvate thereof, as hereinbefore defined for use in therapy.

In a further aspect, the present invention provides the use of a compound of formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substance, which may also act as diluents, flavoring agents, solubilizers, lubricants, suspending agents, binders, or tablet-disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I as defined above for the manufacture of a medicament.

Also within the scope of the invention is the use of any compound of formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the formula I above, is administered to a patient in need of such therapy.

Additionally, there is provided a pharmaceutical composition comprising a compound of Formula I, or a pharmaceutically acceptable salt thereof, in association with a pharmaceutically acceptable carrier.

Particularly, there is provided a pharmaceutical composition comprising a compound of Formula I, or a pharmaceutically acceptable salt thereof, in association with a pharmaceutically acceptable carrier for therapy, more particularly for therapy of pain.

Further, there is provided a pharmaceutical composition comprising a compound of Formula I, or a pharmaceutically acceptable salt thereof, in association with a pharmaceutically acceptable carrier use in any of the conditions discussed above.

Another aspect of the invention is a method of preparing the compounds of the present invention.

In one embodiment, the method of the invention is a method for preparing a compound of formula I,

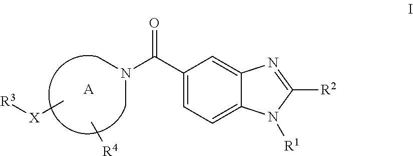

comprising the step of reacting a compound of formula II,

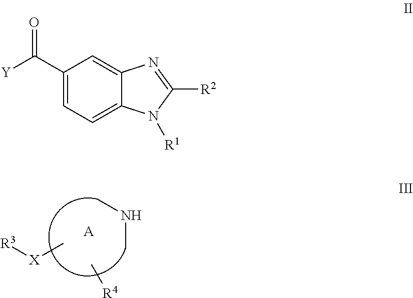

with a compound of formula III, optionally in the presence of a base, such as DIPEA or triethylamine, a coupling agent such as HATU, a solvent such as DMF, wherein Y is selected from Cl, Br, F and OH; and X, A, R¹, R², R³ and R⁴ are defined as above.

Compounds of the present invention may be prepared according to the synthetic routes as depicted in Schemes 1-3.

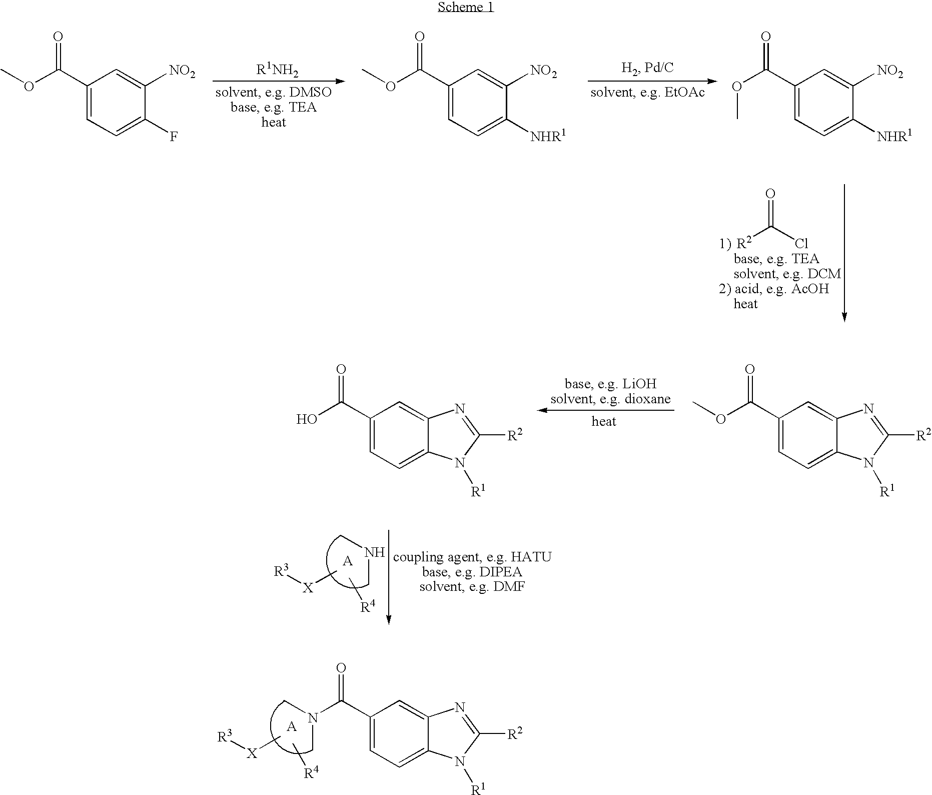

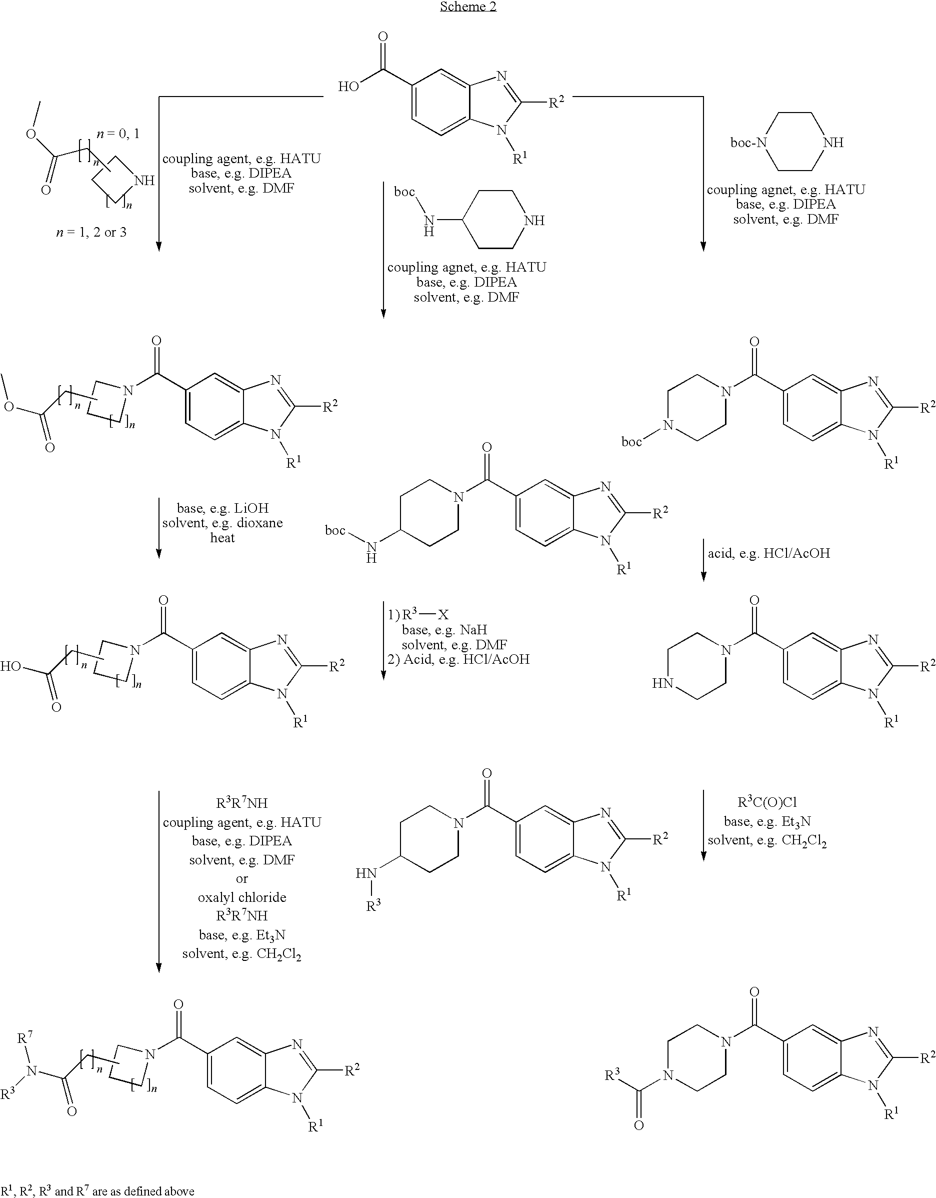

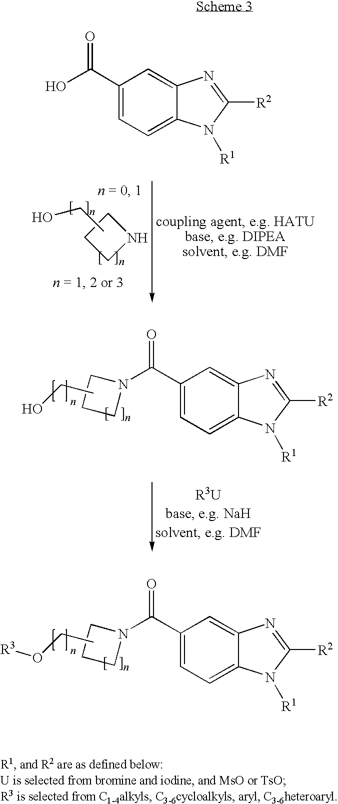

### Biological Evaluation

hCB₁ and hCB₂ Receptor Binding

Human CB₁ receptor from Receptor Biology (hCB₁) or human CB₂ receptor from BioSignal (hCB₂) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed into 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ and hCB₂ GTPγS Binding

Human CB₁ receptor from Receptor Biology (hCB₁) or human CB₂ receptor membranes (BioSignal) are thawed at 37° C., passed 3 times through a 25-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the invention are evaluated from 10-point dose-response curves done in 300 μl with the appropriate amount of membrane protein and 100000-130000 dpm of GTPg³⁵S per well (0.11-0.14 nM). The basal and maximal stimulated binding is determined in absence and presence of 1 μM (hCB₂) or 10 μM (hCB₁) Win 55,212-2 respectively. The membranes are pre-incubated for 5 minutes with 56.25 μM (hCB2) or 112.5 μM (hCB₁) GDP prior to distribution in plates (15 μM (hCB₂) or 30 μM (hCB₁) GDP final). The plates are vortexed and incubated for 60 minutes at room temperature, filtered on Unifilters GF/B (presoaked in water) with the Tomtec or Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) after adding 65 μl/well of MS-20 scintillation liquid. Antagonist reversal studies are done in the same way except that (a) an agonist dose-response curve is done in the presence of a constant concentration of antagonist, or (b) an antagonist dose-response curve is done in the presence of a constant concentration of agonist.

Based on the above assays, the dissociation constant (Ki) for a particular compound of the invention towards a particular receptor is determined using the following equation:

Ki=IC₅₀/(1+[rad]/Kd),

Wherein IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Using the above-mentioned assays, the Ki towards human CB₁ receptors for certain exemplified compounds of the invention is measured to be in the range of 16-3570 nM. The EC₅₀ towards human CB₁ receptors for certain exemplified compounds of the invention is measured to be. in the range of about 16-1768 nM. The Eₘₐₓ towards human CB₁ receptors for certain exemplified compounds of the invention is measured to be in the range of about 112-139%.

The following table shows certain biological activities for some of the exemplified compounds.

| | hCB1 | hCB1 | hCB1 |
|---|---|---|---|
| Compoud | Ki (nM) | EC50 | Emax (%) |
| 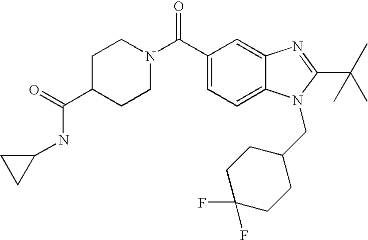 | 19.1720 | 16.7450 | 114.8700 |
| 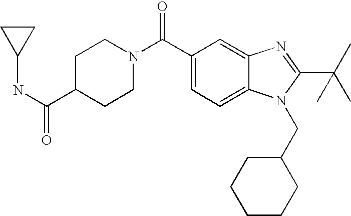 | 23.7980 | 37.7080 | 134.2800 |
| 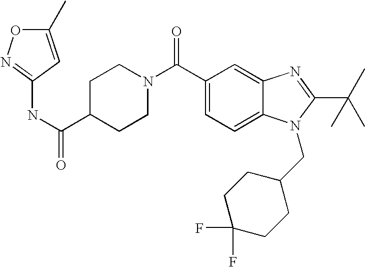 | N/A | 1768.1210 | 132.3200 |

### EXAMPLES

The invention will further be described in more detail by the following Examples which describe methods whereby compounds of the present invention may be prepared, purified, analyzed and biologically tested, and which are not to be construed as limiting the invention.

### Example 1

### 1-({2-tert-Butyl-1-[(4,4-difluorocyclohexyl)methyl]-1H-benzimidazol-5-yl}carbonyl)-N-methylazetidine-3-carboxamide

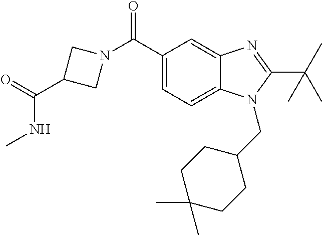

### Step A: 1-({2-tert-Butyl-1-[(4,4-difluorocyclohexyl)methyl]-1H-benzimidazol-5-yl}carbonyl)-N-methylazetidine-3-carboxamide

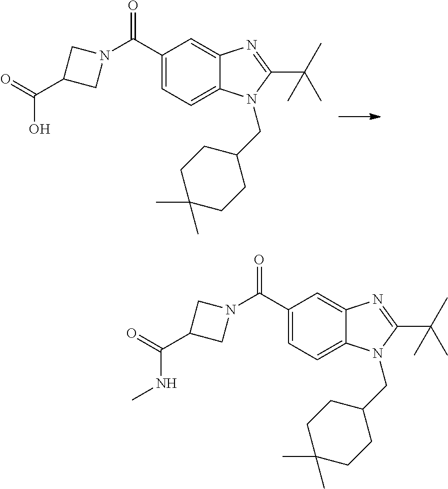

To a mixture of 1-({2-tert-butyl-1-[(4,4-difluorocyclohexyl)methyl]-1H-benzimidazol-5-yl}carbonypazetidine-3-carboxylic acid (for preparation see following steps B to G) (0.030 g, 0.069 mmol) and DIPEA (18 μL, 0.10 mmol) in DMF (5 mL) were added HATU (0.032 g, 0.084 mmol) and a 2M solution of methylamine in THF (0.042 mL, 0.084 mmol). The mixture was stirred at room temperature for 0.5 h. The solvent was removed under reduced pressure. DCM was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 10-70% CH₃CN/H₂O and lyophilized to afford the title compound as the corresponding TFA salt. Yield: 15 mg (39%). ¹H NMR (400 MHz, METHANOL-D₄) δ 1.48-1.84 (m, 6H), 1.67 (s, 9H), 1.99-2.11 (m, 2H), 2.20-2.33 (m, 1H), 2.75 (s, 3H), 3.41-3.50 (m, 1H), 4.20-4.26 (m, 1H), 4.35 (t, J=9.57 Hz, 1H), 4.43-4.52 (m, 2H), 4.55 (d, J=7.62 Hz, 2H), 7.82 (dd, J=8.79, 1.56 Hz, 1H), 7.96 (d, J=8.59 Hz, 1H), 8.00-8.02 (m, 1H); MS (ESI) (M+H)⁺447.3; Anal. Calcd for C₂₄H₃₂N₄O₂F₂+1.8 TFA+1.5 H₂O: C, 48.84; H, 5.46; N, 8.25. Found: C, 48.83; H, 5.38; N, 8.51.

### Step B: Methyl 4-{[(4,4-difluorocyclohexyl)methyl]amino}-3-nitrobenzoate

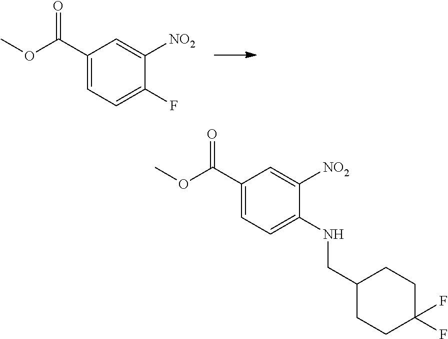

To a mixture of methyl 4-fluoro-3-nitrobenzoate (2.0 g, 10 mmol) and DMSO (40 mL) were added DIPEA (5.2 mL, 30 mmol) and [(4,4-difluorocyclohexyl)methyl]amine HCl salt (2.2 g, 12 mmol). The mixture was stirred overnight at 75° C. DCM was added to the reaction mixture and the organic layer was washed once with a 5% aqueous KHSO₄ solution. The aqueous layer was extracted twice with CH₂Cl₂. The combined organic layer was washed once with a saturated aqueous NaHCO₃ solution, once with brine and dried over anhydrous Na₂SO₄. CH₂Cl₂ was removed under reduced pressure. The resulting residue was purified by column chromatography on silica gel (20:80 to 30:70 EtOAc/hexanes) to afford a yellow solid. Yield: 3.0 g (92%). ¹H NMR (400 MHz, CHLOROFORM-D) δ 1.37-1.50 (m, 2H), 1.68-1.87 (m, 3H), 1.90-1.99 (m, 2H), 2.11-2.23 (m, 2H), 3.29 (dd, J=6.84, 5.66 Hz, 2H), 3.91 (s, 3H), 6.86 (d, J=9.18 Hz, 1H), 8.07 (ddd, J=9.03, 2.10, 0.59 Hz, 1H), 8.43-8.49 (m, 1H), 8.90 (d, J=2.15 Hz, 1H).

### Step C: Methyl 3-amino-4-{[(4,4-difluorocycloheryl)methyl]amino}benzoate

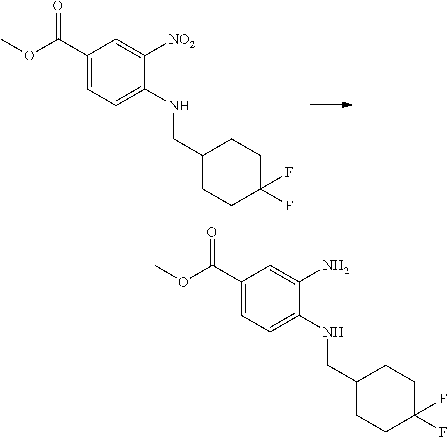

To a mixture of 10% Pd/C (catalytic amount) and ethyl acetate (˜10 mL) was added a solution of methyl 4-{[(4,4-difluorocyclohexyl)methyl]amino}-3-nitrobenzoate (3.0 g, 9.2 mmol) in ethyl acetate (˜50 mL) at room temperature. The mixture was stirred for 2 days at room temperature under a hydrogen atmosphere (50 psi). The mixture was filtered through a pad of Celite. Ethyl acetate was removed at reduced pressure. Yield: 2.2 g (88%). ¹H NMR (400 MHz, CHLOROFORM-D) δ 1.31-1.46 (m, 2H), 1.60-1.83 (m, 6H), 1.88-1.97 (m, 2H), 2.07-2.20 (m, 2H), 3.11 (d, J=6.84 Hz, 2H), 3.85 (s, 3H), 6.57 (d, J=8.40 Hz, 1H,) 7.42 (d, J=1.95 Hz, 1H), 7.59 (dd, J=8.40, 1.95 Hz, 1H).

### Step D: Methyl 2-tert-butyl-1-[(4,4-difluorocyclohexl)methyl]-1H-benzimidazole-5-carboxylate

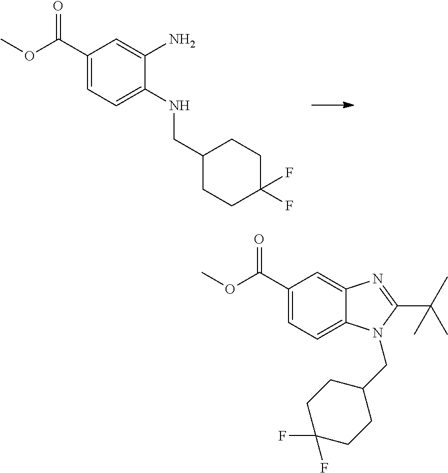

To a mixture of methyl 3-amino-4-{[(4,4-difluorocyclohexyl)methyl]amino}benzoate (2.4 g, 8.1 mmol) and dichloromethane (50 mL) were added DMAP (1.5 g, 12 mmol) and trimethylacetyl chloride (1.1 mL, 8.9 mmol). The mixture was stirred at room temperature for 4 h. DCM was added to the reaction mixture and the organic layer was washed once with a saturated aqueous NaHCO₃ solution, once with brine and dried over anhydrous Na₂SO₄. CH₂Cl₂ was removed under reduced pressure. The resulting residue was dissolved in 60 mL of acetic acid. The mixture was heated in a Personal Chemistry microwave instrument at 150° C. for 3 hours. Acetic acid was removed under reduced pressure. DCM was added to the resulting residue and the organic layer was washed once with a saturated aqueous NaHCO₃ solution, once with brine and dried over anhydrous Na₂SO₄. CH₂Cl₂ was removed under reduced pressure. The resulting residue was dissolved in 10 mL of ethyl acetate. The insoluble part was filtered and appeared to be the uncyclized compound. The soluble part was purified by flash chromatography on silica gel (5:95 to 10:90 Et₂O/CH₂Cl₂). The combined uncyclized portion recovered from the filtration and the column was dissolved in acetic acid again and placed in the microwave at 150° C. for 3 h. The work-up was made as described above and the product was purified in the same way. The reaction was done following this procedure until no more uncyclized material could be observed. Yield: 1.15 g (34%). ¹H NMR (400 MHz, CHLOROFORM-D) δ 1.40-1.75 (m, 6H), 1.57 (s, 9H), 2.07-2.20 (m, 3H), 3.93 (s, 3H), 4.24 (d, J=7.42 Hz, 2H), 7.32 (dd, J=8.50, 0.49 Hz, 1H), 7.96 (dd, J=8.59, 1.56 Hz, 1H), 8.48 (d, J=1.17 Hz, 1H).

### Step E: 2-tert-Butyl-1-[(4,4-difluorocyclohexyl)methyl]-1H-benzimidazole-5-carboxylic acid

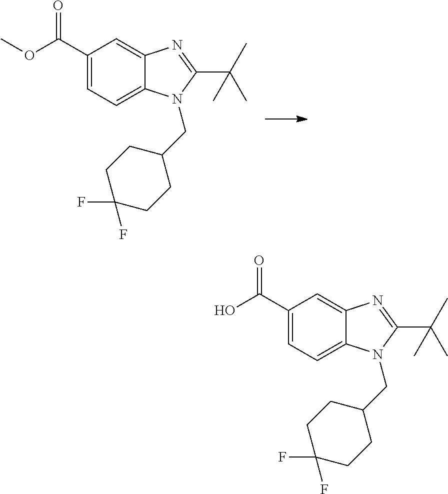

Methyl 2-tert-butyl-1-[(4,4-difluorocyclohexyl)methyl]-1H-benzimidazole-5-carboxylate (1.15 g, 3.16 mmol) was dissolved in 80 mL of a 1:1 mixture of 1M aqueous LiOH and dioxane (1:1). The mixture was stirred at 75° C. for 3 h. The mixture was acidified to pH 5-6 with a 5% aqueous KHSO₄ solution. The mixture was extracted twice with Et₂O. The organic layer was washed once with brine and dried over anhydrous Na₂SO₄. Et₂O was removed at reduced pressure. The product was directly used for the next step. Yield: 1.0 g (95%). ¹H NMR (400 MHz, CHLOROFORM-D) δ 1.44-1.77 (m, 6H), 1.59 (s, 9H), 2.08-2.21 (m, 3H), 4.26 (d, J=7.42 Hz, 2H), 7.36 (d, J=8.59 Hz, 1H), 8.04 (dd, J=8.50, 1.66 Hz, 1H), 8.58-8.59 (m, 1H).

### Step F: Methyl 1-({2-tert-butyl-1-[(4,4-difluorocyclohexyl)methyl]-1H-benzimidazol-5-yl}carbonyl)azetidine-3-carboxylate

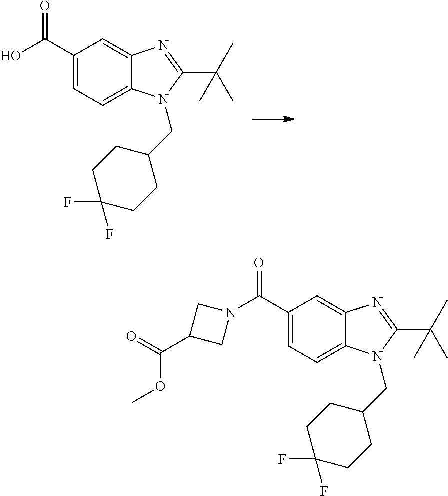

To a mixture of 2-tert-butyl-1-[(4,4-difluorocyclohexyl)methyl]-1H-benzimidazole-5-carboxylic acid (0.130 g, 0.371 mmol), DIPEA (162 μL, 0.927 mmol) and DMF (5 mL) were added HATU (0.155 g, 0.408 mmol) and methyl azetidine-3-carboxylate HCl salt (0.062 g, 0.41 mmol). The mixture was stirred at room temperature overnight. Partial consumption of the starting material was observed by LC-MS. Additional methyl azetidine-3-carboxylate HCl salt (0.030, 0.20 mmol), DIPEA (180 μL, 1.03 mmol) and HATU (0.150 g, 0.395 mmol) were added. The mixture was stirred at room temperature for 1 h. The solvent was removed under reduced pressure. CH₂Cl₂ was added to the resulting residue and the organic layer was washed once with saturated aqueous NaHCO₃ solution, brine and dried over anhydrous Na₂SO₄. CH₂Cl₂ was removed under reduced pressure. The resulting residue was purified by column chromatography on silica gel using 100% ethyl acetate. Yield: 166 mg (99%). ¹H NMR (400 MHz, CHLOROFORM-D) δ 1.34-1.76 (m, 6H), 1.57 (s, 9H), 2.07-2.21 (m, 3H), 3.44-3.54 (m, 1H), 3.77 (s, 3H), 4.24 (d, J=7.42 Hz, 2H), 4.31-4.65 (m, 4H), 7.35 (d, J=8.59 Hz, 1H), 7.68 (dd, J=8.40, 1.56 Hz, 1H), 7.94 (d, J=1.37 Hz, 1H).

### Step G: 1-({2-tert-Butyl-1-[(4,4-difluorocyclohexyl)methyl]-1H-benzimidazol-5-yl}carbonyl)azetidine-3-carboxylic acid

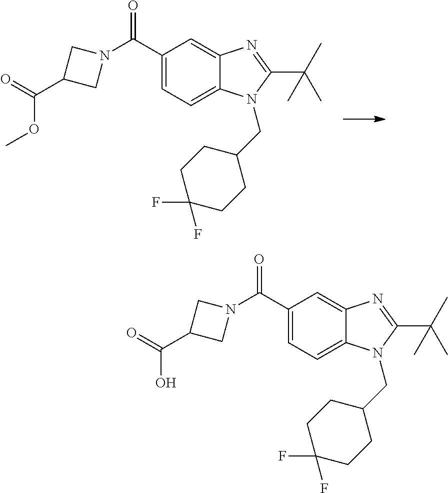

Methyl 1-({2-tert-butyl-1-[(4,4-difluorocyclohexyl)methyl]-1H-benzimidazol-5-yl}carbonyl)azetidine-3-carboxylate (0.166 g, 0.371 mmol) was dissolved in 10 mL of a 1:1 mixture of 1M aqueous LiOH and dioxane. The mixture was stirred at 75° C. for 2 h. The mixture was acidified to pH 5-6 with a 5% aqueous KHSO₄ solution. The mixture was extracted twice with Et₂O. The organic layer was washed once with brine and dried over anhydrous MgSO₄. Et₂O was removed at reduced pressure. The product was directly used for the next step. Yield: 122 mg (76%). MS (ESI) m/z 434.1 (M+H)⁺.

### Example 2

### 1-({2-tert-Butyl-1-[(4,4-difluorocyclohexyl)methyl]-1H-benzimidazol-5-yl}carbonyl)-N-cyclopropylpiperidine-3-carboxamide

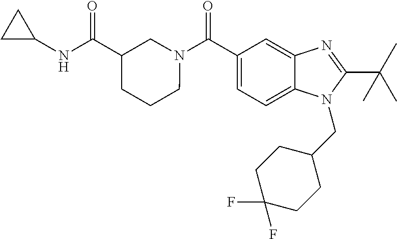

### Step A: 1-({2-tert-Butyl-1-[(4,4-difluorocyclohexyl)methyl]-1H-benzimidazol-5-yl}carbonyl)-N-cyclopropylpiperidine-3-carboxamide

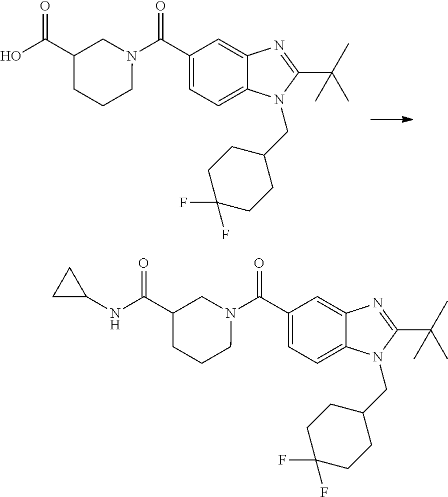

To a mixture of 1-({2-tert-butyl-1-[(4,4-difluorocyclohexyl)methyl]-1H-benzimidazol-5-yl}carbonyl)piperidine-3-carboxylic acid (for preparation see following steps B to C) (0.047 g, 0.10 mmol), DIPEA (35 μL, 0.20 mmol) and DMF (5 mL) were added HAW (0.043 g, 0.11 mmol) and cyclopropylamine (8 μL, 0.11 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 15-60% CH₃CN/H₂O and lyophilized to afford the title compound as the, corresponding TFA salt. Yield: 15 mg (24%). ¹H NMR (400 MHz, METHANOL-D₄) δ 0.27-0.40 (m, 1H), 0.43-0.53 (m, 1H), 0.58-0.77 (m, 2H), 1.49-1.84 (m, 8H), 1.67 (s, 9H), 1.89-2.00 (m, 1H), 2.00-2.12 (m, 2H), 2.19-2.34 (m, 2H), 2.38-2.55 (m, 1H), 2.62-2.71 (m, 1H), 3.08-3.40 (m, 3H), 3.53-3.68 (m, 1H), 4.30-4.47 (m, 1H), 4.55 (d, J=7.42 Hz, 2H), 7.59 (dd, J=8.69, 1.46 Hz, 1H), 7.77 (s, 1H), 7.96 (d, J=8.59 Hz, 1H); MS (ESI) (M+H)⁺ 501.3; Anal. Calcd for C₂₈H₃₈N₄O₂F₂+1.8 TFA+0.8 H₂O: C, 52.69; H, 5.79; N, 7.78. Found: C, 52.77; H, 5.93; N, 7.41.

### Step B: Ethyl 1-({2-tert-butyl-1-[(4,4-difluorocyclohexyl)methyl]-1H-benzimidazol-5-yl}carbonyl)piperidine-3-carboxylate

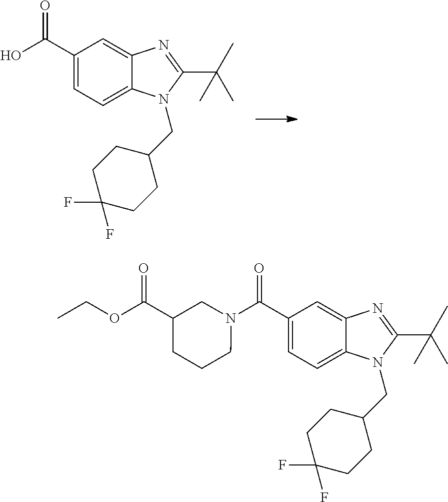

To a mixture of 2-tert-butyl-1-[(4,4-difluorocyclohexyl)methyl]-1H-benzimidazole-5-carboxylic acid (for preparation see Example 1) (0.103 g, 0.294 mmol), DIPEA (102 μL, 0.588 mmol) and DMF (5 mL) were added HATU (0.123 g, 0.323 mmol) and ethyl nipecotate (50 μL, 0.32 mmol) at 0° C. The mixture was slowly allowed to room temperature and stirred for 3 h. The solvent was removed under reduced pressure. CH₂Cl₂ was added to the resulting residue and the organic layer was washed once with saturated aqueous NaHCO₃ solution, brine and dried over anhydrous Na₂SO₄. CH₂Cl₂ was removed under reduced pressure. The resulting residue was purified by column chromatography on silica gel using 100% ethyl acetate as the mobile phase. Yield: 101 mg (70%). MS (ESI) (M+H)⁺490.3.

### Step C: 1-({2-tert-Butyl-1-[(4,4-difluorocyclohexyl)methyl]-1H-benzimidazol-5-yl}carbonyl)piperidine-3-carboxylic acid

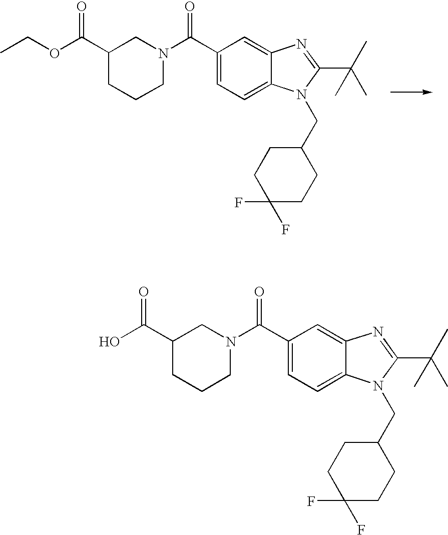

Ethyl 1-({2-tert-butyl-1-[(4,4-difluorocyclohexyl)methyl]-1H-benzimidazol-5-yl}carbonyl)piperidine-3-carboxylate (0.101 g, 0.206 mmol) was dissolved in 10 mL of a 1:1 mixture of 1M aqueous LiOH and dioxane. The mixture was stirred at 75° C. for 2 h. The mixture was acidified to pH 5-6 with a 5% aqueous KHSO₄ solution. The mixture was extracted twice with Et₂O. The organic layer was washed once with brine and dried over anhydrous Na₂SO₄. Et₂O was removed at reduced pressure. The product was directly used for the next step. Yield: 95 mg (99%). MS (ESI) m/z 462.3 (M+H)⁺.

### Example 3

### 1-({2-tert-Butyl-1-[(4,4-difluorocyclohexyl)methyl]-1H-benzimidazol-5-yl}carbonyl)-N-ethylpiperidine-3-carboxamide

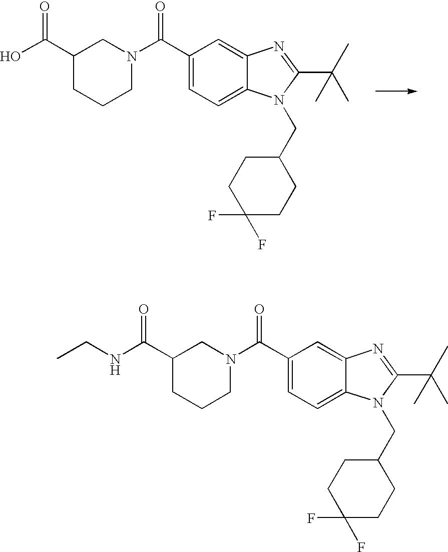

To a mixture of 1-({2-tert-butyl-1-[(4,4-difluorocyclohexyl)methyl]-1H-benzimidazol-5-yl}carbonyl)piperidine-3-carboxylic acid (for preparation see Example 2) (0.047 g, 0.10 mmol), DIPEA (35 μL, 0.20 mmol) and DMF (5 mL) were added HATU (0.043 g, 0.11 mmol) and a 2M solution of ethylamine in THF (56 μL, 0.11 mmol). The mixture was stirred at room temperature for 3 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 10 mg (16%). ¹H NMR (400 MHz, METHANOL-D₄) δ 0.94-1.17 (m, 2H), 1.45-1.83 (m, 8H), 1.65 (s, 9H), 1.91-2.21 (m, 3H), 2.17-2.52 (m, 2H), 2.91-3.38 (m, 6H), 3.56-3.73 (m, 1H), 4.36-4.59 (m, 1H), 4.51 (d, J=7.42 Hz, 2H), 7.54 (d, J=9.18 Hz, 1H), 7.75 (s, 1H), 7.90 (d, J=8.59 Hz, 1H); MS (ESI) (M+H)⁺489.3.

### Example 4

### 1-({2-tert-Butyl-1-[(4,4-difluorocyclohexyl)methyl]-1H-benzimidazol-5-yl}carbonyl)-N-cyclopropylpiperidine-4-carboxamide

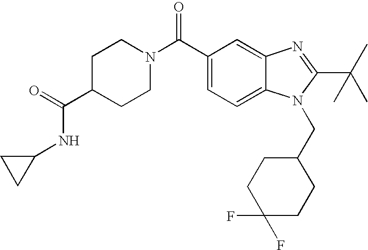

### Step A: 1-({2-tert-Butyl-1-[(4,4-difluorocyclohexyl)methyl]-1H-benzimidazol-5-yl}carbonyl)-N-cyclopropylpiperidine-4-carboxamide

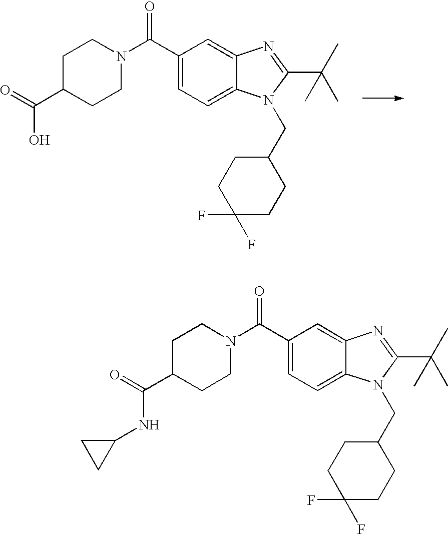

To a mixture of 1-({2-tert-butyl-1-[(4,4-difluorocyclohexyl)methyl]-1H-benzimidazol-5-yl}carbonyl)piperidine-4-carboxylic acid (for preparation see following steps B to C) (0.043 g, 0.093 mmol), DIPEA (32 μL, 0.186 mmol) and DMF (5 mL) were added HATU (0.039 g, 0.10 mmol) and cyclopropylamine (7 μL, 0.10 mmol) at 0° C. The mixture was stirred at 0° C. for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 23 mg (40%). ¹H NMR (400 MHz, METHANOL-D₄) δ 0.42-0.47 (m, 2H), 0.67-0.74 (m, 2H), 1.49-1.93 (m, 11H), 1.68 (s, 9H), 2.00-2.12 (m, 2H), 2.20-2.33 (m, 1H), 2.38-2.49 (m, 1H), 2.59-2.67 (m, 1H), 2.87-3.01 (m, 1H), 3.07-3.22 (m, 1H), 3.65-3.79 (m, 1H), 4.57 (d, J=7.42 Hz, 2H), 4.60-4.71 (m, 1H), 7.61 (dd, J=8.59, 1.37 Hz, 1H), 7.77-7.80 (m, 1H), 8.00 (d, J=8.59 Hz, 1H); MS (ESI) (M+H)⁺501.3; Anal. Calcd for C₂₈H₃₈N₄O₂F₂+1.5 TFA+0.3 H₂O: C, 54.99; H, 5.97; N, 8.27. Found: C, 55.04; H, 5.94; N, 8.08.

### Step B: Methyl 1-({2-tert-butyl-1-[(4,4-difluorocyclohexyl)methyl]-1H-benzimidazol-5-yl}carbonyl)piperidine-4-carboxylate

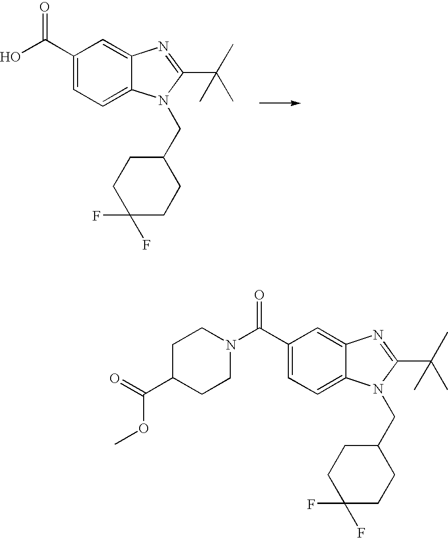

To a mixture of 2-tert-butyl-1-[(4,4-difluorocyclohexyl)methyl]-1H-benzimidazole-5-carboxylic acid (for preparation see Example 1) (0.050 g, 0.14 mmol), DIPEA (50 μL, 0.29 mmol) and DMF (3 mL) were added HATU (0.060 g, 0.16 mmol) and methyl isonipecotate (21 μL, 0.16 mmol). The mixture was stirred at room temperature for 4 h. The solvent was removed under reduced pressure. CH₂Cl₂ was added to the resulting residue and the organic layer was washed once with saturated aqueous NaHCO₃ solution, brine and dried over anhydrous Na₂SO₄. CH₂Cl₂ was removed under reduced pressure. The resulting residue was purified by column chromatography on silica gel using 100% ethyl acetate. Yield: 52 mg (80%). ¹H NMR (400 MHz, CHLOROFORM-D) δ 1.35-1.83 (m, 10H), 1.57 (s, 9H), 1.86-2.03 (m, 2H), 2.08-2.19 (m, 3H), 2.55-2.64 (m, 1H), 3.02-3.14 (m, 2H), 3.71 (s, 3H), 4.23 (d, J=7.42 Hz, 2H), 7.31-7.38 (m, 2H), 7.74-7.76 (m, 1H).

### Step C: 1-({2-tert-Butyl-1-[(4,4-difluorocyclohexyl)methyl]-1H-benzimidazol-5-yl}carbonyl)piperidine-4-carboxylic acid

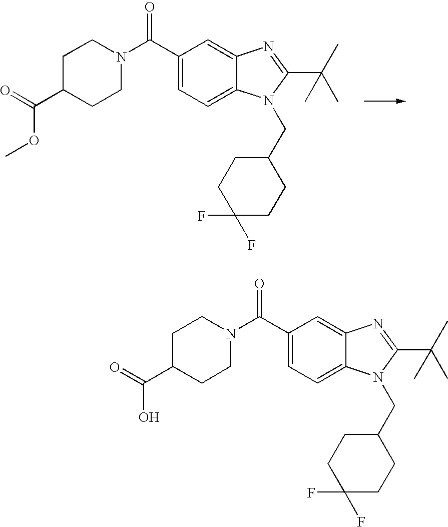

Methyl 1-({2-tert-butyl-1-[(4,4-difluorocyclohexyl)methyl]-1H-benzimidazol-5-yl}carbonyl)piperidine-4-carboxylate (0.052 g, 0.11 mmol) was dissolved in 5 mL of a 1:1 mixture of 1M aqueous LiOH and dioxane. The mixture was stirred at 75° C. for 2 h. The mixture was acidified to pH 5-6 with a 5% aqueous KHSO₄ solution. The mixture was extracted twice with Et₂O. The organic layer was washed once with brine and dried over anhydrous Na₂SO₄. Et₂O was removed at reduced pressure. The product was directly used for the next step. Yield: 43 mg (85%). ¹H NMR (400 MHz, CHLOROFORM-D) δ 1.40-1.76 (m, 9H), 1.58 (s, 9H), 1.83-1.98 (m, 2H), 2.02-2.20 (m, 4H), 2.59-2.70 (m, 1H), 2.94-3.08 (m, 2H), 4.25 (d, J=7.42 Hz, 2H), 7.34-7.39 (m, 1H), 7.44-7.49 (m, 1H), 7.90 (s, 1H).

### Example 5

### 1-({2-tert-Butyl-1-[(4,4-difluorocyclohexyl)methyl]-1H-benzimidazol-5-yl}carbonyl)-N-ethylpiperidine-4-carboxamide

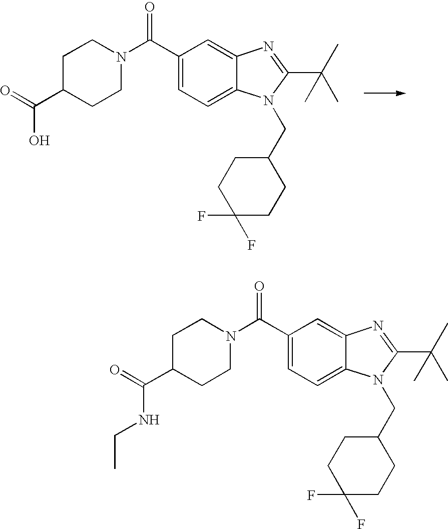

To a mixture of 1-({2-tert-butyl-1-[(4,4-difluorocyclohexyl)methyl]-1H-benzimidazol-5-yl}carbonyl)piperidine-4-carboxylic acid (for preparation see Example 4) (0.043 g, 0.093 mmol), DIPEA (32 μL, 0.186 mmol) and DMF (5 mL) were added HATU (0.039 g, 0.10 mmol) followed by a 2M solution of ethylamine in THF (51 μL, 0.10 mmol). The mixture was stirred at room temperature for 2 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 30 mg (53%). ¹H NMR (400 MHz, METHANOL-D₄) δ 1.09 (t, J=7.32 Hz, 3H), 1.48-1.83 (m, 10H), 1.66 (s, 9H), 1.83-1.95 (m, 1H), 1.99-2.11 (m, 2H), 2.19-2.33 (m, 1H), 2.43-2.55 (m, 1H), 2.82-3.01 (m, 1H), 3.18 (q, J=7.29 Hz, 2H), 3.67-3.81 (m, 1H), 4.54 (d, J=7.62 Hz, 2H), 4.59-4.73 (m, 1H), 7.57 (dd, J=8.59, 1.37 Hz, 1H), 7.76-7.78 (m, 1H), 7.94 (d, J=8.59 Hz, 1H); MS (ESI) (M+H)⁺489.3.

### Example 6

### 1-({2-tert-Butyl-1-[(4,4-difluorocyclohexyl)methyl]-1H-benzimidazol-5-yl}carbonyl)-N-methylpiperidine-4-carboxamide

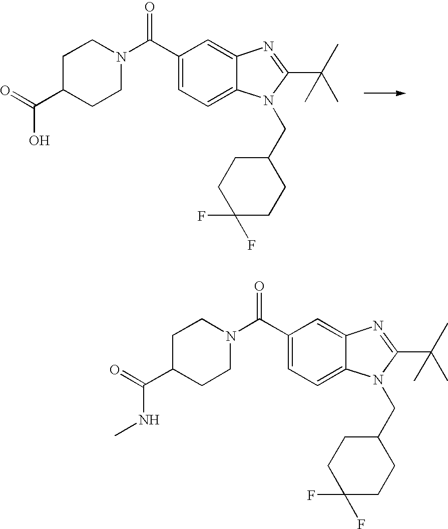

To a mixture of 1-({2-tert-butyl-1-[(4,4-difluorocyclohexyl)methyl]-1H-benzimidazol-5-yl}carbonyl)piperidine-4-carboxylic acid (for preparation see Example 4) (0.043 g, 0.093 mmol), DIPEA (32 μL, 0.186 mmol) and DMF (5 mL) were added HATU (0.039 g, 0.10 mmol) followed by a 2M solution of methylamine in THF (51 μL, 0.10 mmol). The mixture was stirred at room temperature for 2 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 23 mg (42%). ¹H NMR (400 MHz, METHANOL-D₄) δ 1.45-1.83 (m, 9H), 1.65 (s, 9H), 1.82-1.96 (m, 1H), 1.97-2.12 (m, 2H), 2.16-2.34 (m, 1H), 2.42-2.54 (m, 1H), 2.70 (s, 3H), 2.87-3.02 (m, 1H), 3.08-3.22 (m, 1H), 3.68-3.85 (m, 1H), 4.51 (d, J=7.62 Hz, 2H), 4.58-4.73 (m, 1H), 7.54 (dd, J=8.40, 1.37 Hz, 1H), 7.76 (d, J=0.78 Hz, 1H), 7.90 (d, J=8.40 Hz, 1H); MS (ESI) (M+H)⁺475.3.

### Example 7

### N-(tert-Butyl)-1-({2-tert-butyl-1-[(4,4-difluorocyclohexyl)methyl]-1H-benzimidazol-5-yl}carbonyl)piperidine-4-carboxamide

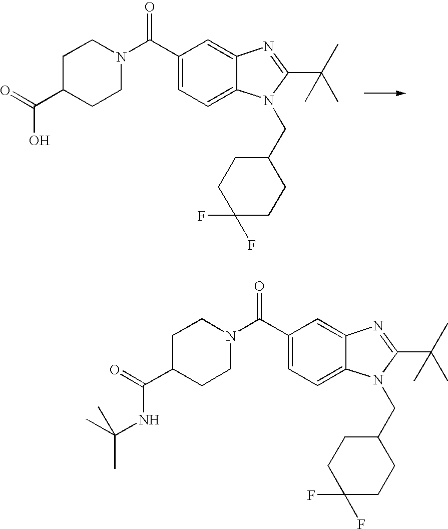

To a mixture of 1-({2-tert-butyl-1-[(4,4-difluorocyclohexyl)methyl]-1H-benzimidazol-5-yl}carbonyl)piperidine-4-carboxylic acid (for preparation see Example 4) (0.047 g, 0.10 mmol), DIPEA (35 μL, 0.20 mmol) and DMF (4 mL) were added HATU (0.046 g, 0.12 mmol) and tert-butylamine (13 μL, 0.12 mmol) at 0° C. The mixture was stirred at rt for 1.5 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% to CH₃CN/H₂O and lyophilized to afford the title compound as the corresponding TFA salt. Yield: 28 mg (44%). ¹H NMR (400 MHz, METHANOL-D₄) δ 1.30 (s, 9H), 1.49-1.92 (m, 8H), 1.67 (s, 9H), 1.99-2.12 (m, 2H), 2.18-2.33 (m, 1H), 2.40-2.51 (m, 1H), 2.82-2.99 (m, 1H), 2.83-2.99 (m, 1H), 3.06-3.23 (m, 2H), 3.65-3.78 (m, 1H), 4.56 (d, J=7.62 Hz, 2H), 4.60-4.72 (m, 1H), 7.60 (dd, J=8.59, 1.56 Hz, 1H), 7.78 (d, J=0.98 Hz, 1H), 7.92-8.01 (m, 1H); MS (ESI) (M+H)⁺517.1.

### Example 8

### 1-({2-tert-Butyl-1-[(4,4-difluorocyclohexyl)methyl]-1H-benzimidazol-5-yl}carbonyl)-N-cyclobutylpiperidine-4-carboxamide

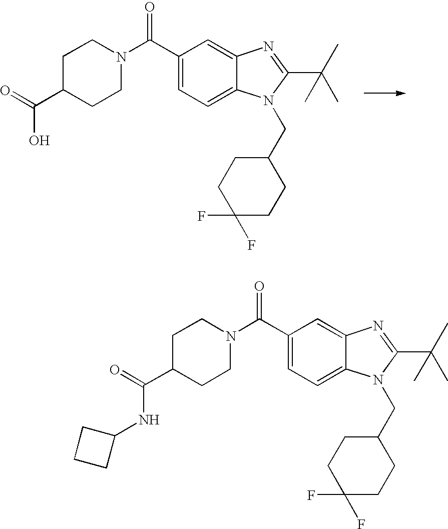

To a mixture of 1-({2-tert-butyl-1-[(4,4-difluorocyclohexyl)methyl]-1H-benzimidazol-5-yl}carbonyl)piperidine-4-carboxylic acid (for preparation see Example 4) (0.050 g, 0.108 mmol), DIPEA (28 μL, 0.162 mmol) and DMF (3 mL) were added HATU (0.049 g, 0.130 mmol) and cyclobutylamine (11 μL, 0.130 mmol). The mixture was stirred at room temperature for 1.5 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 70 mg (99%). ¹H NMR (400 MHz, METHANOL-D₄) δ 1.51-1.66 (m, 4H), 1.68 (s, 9H), 1.69-1.80 (m, 7H), 1.82-1.98 (m, 3H), 2.00-2.12 (m, 3H), 2.19-2.32 (m, 3H), 2.41-2.51 (m, 1H), 2.94 (s, 1H), 3.16 (s, 1H), 3.71 (s, 1H), 4.20-4.32 (m, 1H), 4.57 (d, J=7.62 Hz, 2H), 7.62 (dd, J=8.59, 1.56 Hz, 1H), 7.79 (d, J=0.78 Hz, 1H), 8.01 (d, J=8.79 Hz, 1H); MS (ESI) (M+H)⁺515.3.

### Example 9

### 1-({2-tert-Butyl-1-[(4,4-difluorocyclohexyl)methyl]-1H-benzimidazol-5-yl}carbonyl)-N-isoxazol-3-ylpiperidine-4-carboxamide

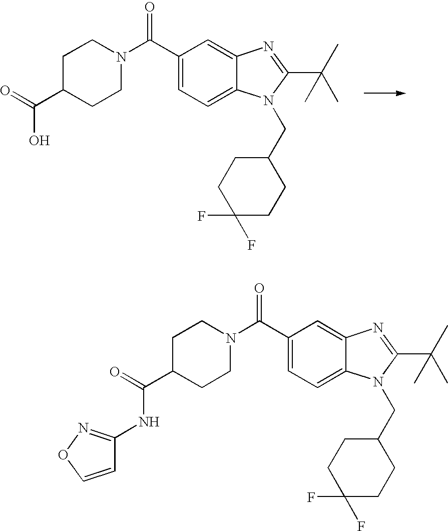

1-({2-tert-Butyl-1-[(4,4-difluorocyclohexyl)methyl]-1H-benzimidazol-5-yl}carbonyl)piperidine-4-carboxylic acid (for preparation see Example 4) (0.034 g, 0.0737 mmol) was dissolved in 5 mL of DCM containing 1 drop of DMF at 0° C. under nitrogen. Oxalyl chloride (0.008 mL, 0.0884 mmol) was added and the solution was stirred at room temperature for 1 h. The solvent was evaporated. The residue was dissolved in 3 mL of DCM and 3-aminoisoxazole (0.011 mL, 0.147 mmol) was added, followed by triethylamine (26 μL, 0.184 mmol). The solution was stirred at room temperature for 2 h. The organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 10 mg (21%). ¹H NMR (400 MHz, METHANOL-D₄) δ 1.51-1.63 (m, 2H), 1.68 (s, 9H), 1.69-1.85 (m, 7H), 2.00-2.13 (m, 3H), 2.27 (s, 1H), 2.75 (s, 1H), 3.04 (s, 1H), 3.22 (s, 1H), 3.77 (s, 1H), 4.56 (d, J=7.62 Hz, 2H), 4.66 (s, 1H), 6.90 (d, J=1.56 Hz, 1H), 7.62 (dd, J=8.59, 1.56 Hz, 1H), 7.80 (d, J=0.78 Hz, 1H), 7.99 (d, J=8.59 Hz, 1H), 8.50 (d, J=1.76 Hz, 1H); MS (ESI) (M+H)⁺ 528.3.

### Example 10

### 1-({2-tert-Butyl-1-[(4,4-difluorocyclohexyl)methyl]-1H-benzimidazol-5-yl}carbonyl)-N-1,3-thiazol-2-ylpiperidine-4-carboxamide

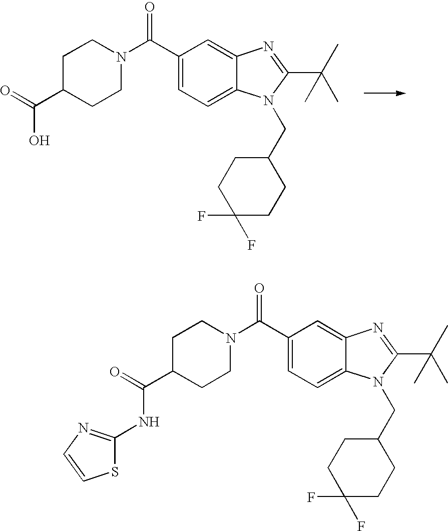

1-({2-tert-Butyl-1-[(4,4-difluorocyclohexyl)methyl]-1H-benzimidazol-5-yl}carbonyl)piperidine-4-carboxylic acid (for preparation see Example 4) (0.050 g, 0.108 mmol) was dissolved in 5 mL of DCM containing 1 drop of DMF at 0° C. under nitrogen. Oxalyl chloride (0.011 mL, 0.130 mmol) was added and the solution was stirred at room temperature for 1 h. The solvent was evaporated. The residue was dissolved in 3 mL of CH₂Cl₂ and 2-aminothiazole (0.032 g, 0.324 mmol) was added, followed by triethylamine (0.045 mL, 0.324 mmol). The solution was stirred at room temperature for 2 h. The organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 26 mg (37%). ¹H NMR (400 MHz, METHANOL-D₄) δ 1.51-1.64 (m, 2H), 1.66-1.70 (m, 9H), 1.70-1.87 (m, 6H), 2.00-2.12 (m, 3H), 2.27 (s, 1H), 2.84 (s, 1H), 3.07 (s, 1H), 3.23 (s, 1H), 3.77 (s, 1H), 4.58 (d, J=7.42 Hz, 2H), 4.67 (s, 1H), 7.11 (d, J=3.32 Hz, 1H), 7.42 (s, 1H,) 7.65 (dd, J=8.69, 1.46 Hz, 1H), 7.82 (d, J=78 Hz, 1H), 8.02 (d, J=8.59 Hz, 1H); MS (ESI) (M+H)⁺544.3; Anal. Calcd for C₂₈H₃₅N₅O₂SF₂+2.2 TFA+0.1 H₂O: C, 48.87; H, 4.73; N, 8.73. Found: C, 48.91; H, 4.67; N, 8.33.

### Example 11

### 1-({2-tert-Butyl-1-[(4,4-difluorocyclohexyl)methyl]-1H-benzimidazol-5-yl}carbonyl)-N-(5-methylisoxazol-3-yl)piperidine-4-carboxamide

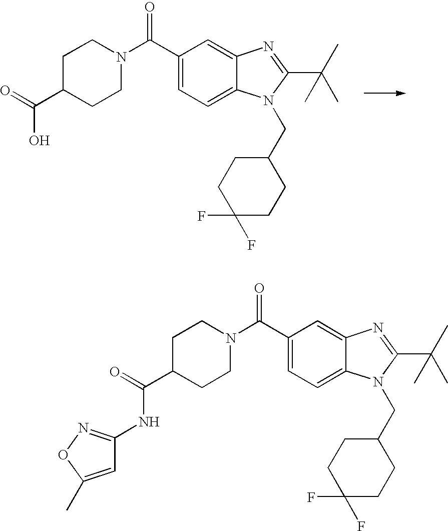

1-({2-tert-Butyl-1-[(4,4-difluorocyclohexyl)methyl]-1H-benzimidazol-5-yl}carbonyl)piperidine-4-carboxylic acid (for preparation see Example 4) (0.050 g, 0.108 mmol) was dissolved in 5 mL of DCM containing 1 drop of DMF at 0° C. under nitrogen. Oxalyl chloride (0.012 mL, 0.130 mmol) was added and the solution was stirred at room temperature for 1 h. The solvent was evaporated. The residue was dissolved in 3 mL of DCM and 3-amino-5-methylisoxazole (0.032 g, 0.324 mmol) was added, followed by triethylamine (0.045 mL, 0.324 mmol) and a catalytic amount of DMAP. The solution was stirred at 50° C. for 3 h. The organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 31 mg (44%); ¹H NMR (400 MHz, METHANOL-D₄) δ 1.51-1.63 (m, 2H), 1.68 (m, 10H), 1.69-1.84 (m, 6H), 1.97-2.11 (m, 3H), 2.22-2.32 (m, 1H), 2.37 (d, J=0.98 Hz, 3H), 2.68-2.78 (m, 1H), 3.03 (s, 1H), 3.21 (s, 1H), 3.75 (s, 1H), 4.56 (d, J=7.42 Hz, 2H), 4.66 (s, 1H), 6.56 (s, 1H), 7.62 (dd, J=8.59, 1.56 Hz, 1H), 7.80 (d, J=0.78 Hz, 1H), 8.00 (d, J=8.59 Hz, 1H); MS (ESI) (M+H)⁺ 542.3; Anal. Calcd for C₂₉H₃₇N₅O₃F₂+1.9 TFA+0.1 H₂O: C, 51.83; H, 5.19; N, 9.21. Found C, 51.91; H, 5.26; N, 9.03.

### Example 12

### 1-({2-tert-Butyl-1-[(4,4-difluorocyclohexyl)methyl]-1H-benzimidazol-5-yl}carbonyl)-N-ethyl-N-methylpiperidine-4-carboxamide

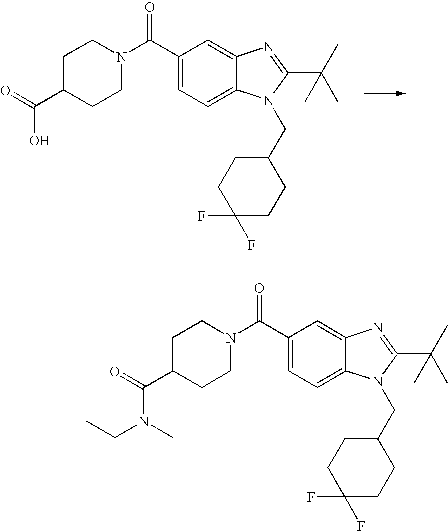

To a mixture of 1-({2-tert-butyl-1-[(4,4-difluorocyclohexyl)methyl]-1H-benzimidazol-5-yl}carbonyl)piperidine-4-carboxylic acid (for preparation see following Example 4) (0.050 g, 0.108 mmol), DIPEA (0.028 mL, 0.162 mmol) and DMF (5 mL) were added HATU (0.049 g, 0.130 mmol) and N-ethylmethylamine (0.011 mL, 0.130 mmol) at rt. The mixture was stirred at rt for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 51 mg (77%); ¹H NMR (400 MHz, METHANOL-D₄) δ 1.08 (t, J=7.13 Hz, 1.5H), 1.21 (t, J=7.13 Hz, 1.5H), 1.51-1.66 (m, 4H), 1.69 (s, 9H), 1.71-1.81 (m, 5H), 1.80-1.93 (m, 1H), 2.01-2.12 (m, 2H), 2.22-2.33 (m, 1H), 2.90 (s, 1.5H), 2.96-3.06 (m, 2H), 3.10 (s, 1.5H), 3.23 (s, 1H), 3.34-3.43 (m, 1H), 3.44-3.52 (m, 1H), 3.71 (s, 1H), 4.58 (d, J=7.62 Hz, 2H), 4.67 (s, 1H), 7.63 (dd, J=8.59, 1.56 Hz, 1H), 7.80 (s, 1H), 8.02 (d, J=9.18 Hz, 1H); MS (ESI) (M+H)⁺ 503.3; Anal. Calcd for C₂₈H₄₀N₄O₂F₂+2.2 TFA+1.4 H₂O: C, 49.97; H, 5.82; N, 7.19. Found: C, 49.90; H, 5.74; N, 7.57.

### Example 13

### 1-({2-tert-Butyl-1-[(4,4-difluorocyclohexyl)methyl]-1H-benzimidazol-5-yl}carbonyl)-N-(cyclopropylmethyl)piperidine-4-carboxamide

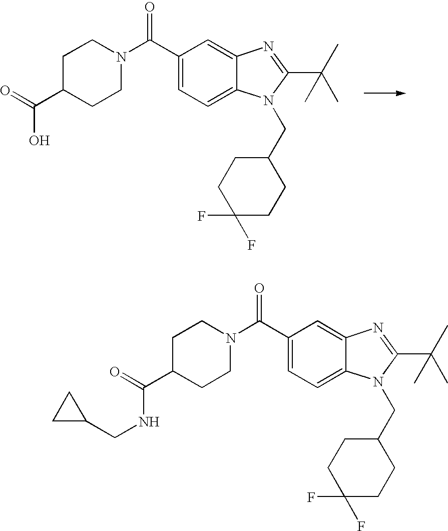

To a mixture of 1-({2-tert-butyl-1-[(4,4-difluorocyclohexyl)methyl]-1H-benzimidazol-5-yl}carbonyl)piperidine-4-carboxylic acid (for preparation see following Example 4) (0.050 g, 0.108 mmol), DIPEA (0.028 mL, 0.162 mmol) and DMF (5 mL) were added HATU (0.049 g, 0.130 mmol) and cyclopropanemethylamine (0.011 mL, 0.130 mmol) at rt. The mixture was stirred at rt for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 41 mg (60%); ¹H NMR (400 MHz, METHANOL-D₄) δ 0.14-0.21 (m, 2H), 0.43-0.51 (m, 2H), 0.89-0.99 (m, 1H), 1.51-1.64 (m, 2H), 1.69 (s, 12H), 1.71-1.81 (m, 4H), 1.90 (s, 1H), 2.01-2.12 (m, 2H), 2.22-2.32 (m, 1H), 2.48-2.58 (m, 1H), 2.95 (s, 1H), 3.02 (d, J=6.84 Hz, 2H), 3.17 (s, 1H), 3.71 (s, 1H), 4.58 (d, J=7.62 Hz, 2H), 4.66 (s, 1H), 7.63 (dd, J=8.69, 1.46 Hz, 1H), 7.79 (d; J=0.98 Hz, 1H), 8.01 (d, J=8.79 Hz, 1H); MS (ESI) (M+H)⁺ 515.3; Anal. Calcd for C₂₉H₄₀N₄O₂F₂+1.9 TFA+2.0 H₂O: C, 51.10; H, 6.05; N, 7.31. Found: C, 51.05; H, 5.95; N, 7.71.

### Example 14

### 1-({2-tert-Butyl-1-[(4,4-difluorocyclohexyl)methyl]-1H-benzimidazol-5-yl}carbonyl)-N-cydopropylpyrrolidine-3-carboxamide

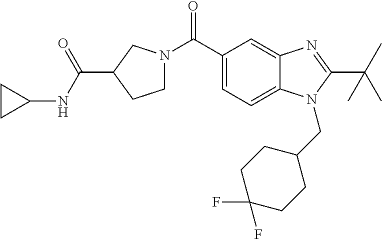

### Step A: 1-({2-tert-Butyl-1-[(4,4-difluorocyclohexyl)methyl]-1H-benzimidazol-5-yl}carbonyl)-N-cyclopropylpyrrolidine-3-carboxamide

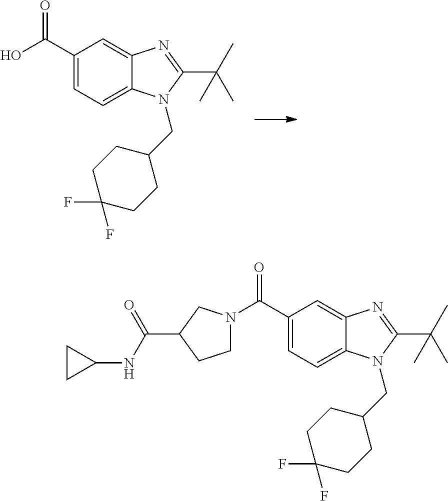

To a mixture of 2-tert-butyl-1-[(4,4-difluorocyclohexyl)methyl]-1H-benzimidazole-5-carboxylic acid (for preparation see Example 1) (0.050 g, 0.14 mmol), DIPEA (62 μL, 0.36 mmol) and DMF (4 mL) were added HATU (0.065 g, 0.17 mmol) and N-cyclopropylpyrrolidine-3-carboxamide HCl salt (for preparation see following steps B and C) (0.037 g, 0.17 mmol) at 0° C. The mixture was stirred at 0° C. for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and is lyophilized to afford the title compound as the corresponding TFA salt. Yield: 12 mg (14%). ¹H NMR (400 MHz, METHANOL-D₄) δ 0.35-0.53 (m, 2H), 0.62-0.76 (m, 2H), 1.47-1.82 (m, 6H), 1.64 (s, 9H), 1.98-2.32 (m, 5H), 2.56-2.71 (m, 1H), 2.56-3.09 (m, 1H), 3.49-3.84 (m, 4H), 4.50 (d, J=7.62 Hz, 2H), 7.62-7.68 (m, 1H), 7.83-7.91 (m, 2H); MS (ESI) (M+H)⁺ 487.0.

### Step B: tert-Butyl 3-[(cyclopropylamino)carbonyl]pyrrolidine-1-carboxylate

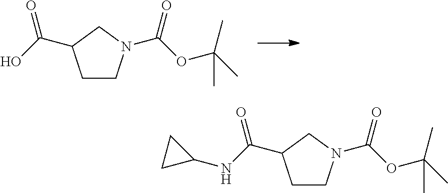

To a mixture of 1-(tert-butoxycarbonyl)pyrrolidine-3-carboxylic acid (0.100 g, 0.465 mmol), DIPEA (121 μL, 0.697 mmol) and DMF (5 mL) were added HATU (0.212 g, 0.557 mmol) and cyclopropylamine (39 μL, 0.557 mmol). The mixture was stirred at room temperature for 1 h. The solvent was removed under reduced pressure. CH₂Cl₂ was added to the resulting residue and the organic layer was washed once with a 5% aqueous KHSO₄ solution, once with saturated aqueous NaHCO₃ solution, brine and dried over anhydrous Na₂SO₄. CH₂Cl₂ was removed under reduced pressure. The resulting product was directly to used for the next step. Yield: 115 mg (97%). MS (ESI) (M−C₄H₉)⁺ 198, (M−C₄H₉O)⁺ 181, (M−C₅H₉O₂)⁺ 153.

### Step C: N-Cyclopropylpyrrolidine-3-carboxamide

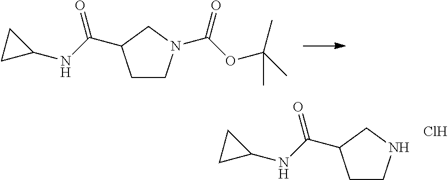

tert-Butyl-3-[(cyclopropylamino)carbonyl]pyrrolidine-1-carboxylate (0.115 g, 0.452 mmol) was dissolved in 5 mL of a 1M solution of HCl in acetic acid. The mixture was stirred at room temperature for 2 h. The solvent was removed under pressure. Et₂O was added to the resulting residue. The product was filtered and placed under the pump. The product was directly used for the next step. Yield: 43 mg (85%). ¹H NMR (400 MHz, METHANOL-D₄) δ 0.45-0.50 (m, 2H), 0.68-0.75 (m, 2H), 2.03-2.13 (m, 1H), 2.20-2.31 (m, 1H), 2.61-2.69 (m, 1H), 3.04-3.13 (m, 1H), 3.31-3.42 (m, 3H), 3.43-3.49 (m, 1H).

### Example 15

### 2-tert-Butyl-1-[(4,4-difluorocyclohexyl)methyl]-5-[(4-methoxypiperidin-1-yl)carbonyl]-1H-benzimidazole

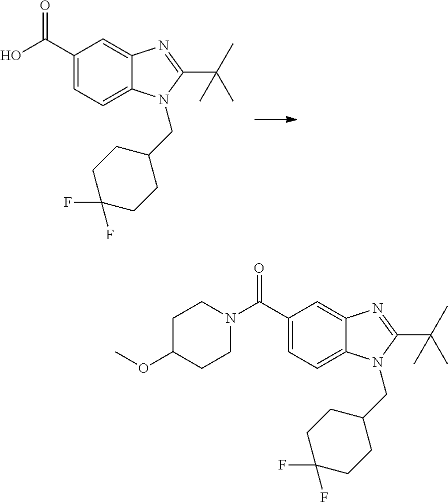

To a mixture of 2-tert-butyl-1-[(4,4-difluorocyclohexyl)methyl]-1H-benzimidazole-5-carboxylic acid (for preparation see Example 1) (0.050 g, 0.143 mmol), DIPEA (0.063 mL, 0.358 mmol) and DMF (5 mL) were added HATU (0.065 g, 0.172 mmol) and 4-methoxypiperidine hydrochloride (0.026 g, 0.172 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 80 mg (99%); ¹H NMR (400 MHz, METHANOL-D₄) δ 1.49-1.63 (m, 4H), 1.66-1.69 (m, 10H), 1.71-1.86 (m, 5H), 1.97-2.11 (m, 3H), 2.21-2.32 (m, 1H), 3.36 (s, 3H), 3.49-3.60 (m, 3H), 4.02 (s, 1H), 4.57 (d, J=7.62 Hz, 2H), 7.61 (dd, J=8.69, 1.46 Hz, 1H), 7.78 (d, J=0.78 Hz, 1H), 7.99 (d, J=8.79 Hz, 1H); MS (ESI) (M+H)⁺ 448.3; Anal. Calcd for C₂₅H₃₅N₃O₂F₂+1.7 TFA+0.5 H₂O: C, 52.45; H, 5.84; N, 6.46. Found: C, 52.37; H, 5.80; N, 6.60.

### Example 16

### 2-tert-Butyl-1-[(4,4-difluorocyclohexyl)methyl]-5-[(4-ethoxypiperidin-1-yl)carbonyl]-1H-benzimidazole

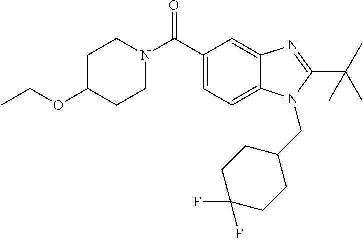

### Step A. 2-tert-Butyl-1-[(4,4-difluorocyclohexyl)methyl]-5-[(4-ethoxypiperidin-1-yl)carbonyl]-1H-benzimidazole

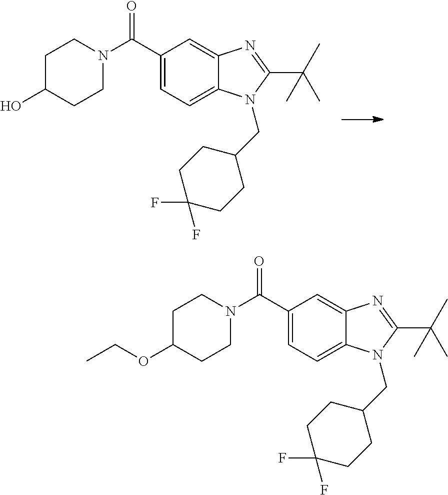

To a solution of NaH (0.012 g, 0.288 mmol) in 2 mL of DMF at 0° C. under nitrogen, a DMF solution (5 mL) of 1-({2-tert-butyl-1-[(4,4-difluorocyclohexyl)methyl]-1H-benzimidazol-5-yl}carbonyl)piperidin-4-ol (for preparation, see Step B) (0.050 g, 0.115 mmol) was added dropwise. The solution was stirred at 0° C. under nitrogen for 30 min. Iodoethane (0.023 mL, 0.288 mmol) was added dropwise and the solution was stirred at it for 3 h. The, reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 55 mg (83%); ¹H NMR (400 MHz, METHANOL-D₄) δ 1.18 (t, J=7.03 Hz, 3H), 1.49-1.63 (m, 2H), 1.68 (s, 10H), 1.70-1.85 (m, 5H), 1.98 (s, 1H), 2.01-2.12 (m, 3H), 2.27 (m, 1H), 3.24 (s, 1H), 3.47-3.60 (m, 3H), 3.60-3.68 (m, 2H), 4.07 (s, 1H), 4.57 (d, J=7.62 Hz, 2H), 7.61 (dd, J=8.69, 1.46 Hz, 1H), 7.78 (d, J=0.78 Hz, 1H), 7.99 (d, J=8.59 Hz, 1H); MS (ESI) (M+H)⁺ 462.3; Anal. Calcd for C₂₆H₃₇N₃O₂F₂+1.7 TFA+0.3 H₂O: C, 53.44; H, 5.99; N, 6.36. Found: C, 53.41; H, 5.87; N, 6.43.

### Step B. 1-({2-tert-Butyl-1-[(4,4-difluorocyclohexyl)methyl]-1H-benzimidazol-5-yl}carbonyl)piperidin-4-ol

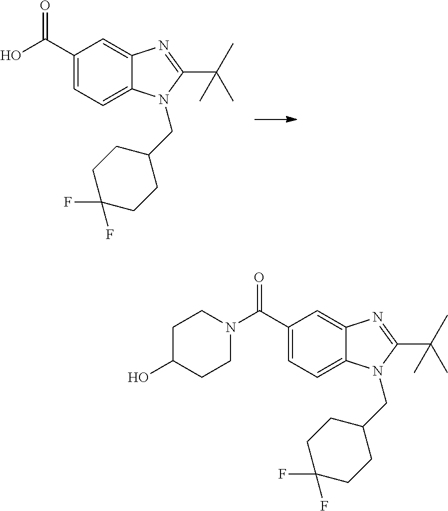

To a mixture of 2-tert-butyl-1-[(4,4-difluorocyclohexyl)methyl]-1H-benzimidazole-5-carboxylic acid (for preparation see Example 1) (0.250 g, 0.713 mmol), DIPEA (0.185 mL, 1.07 mmol) and DMF (8 mL) were added HATU (0.325 g, 0.856 mmol) and 4-hydroxypiperidine (0.086 g, 0.856 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by silica gel flash chromatography using 5% MeOH/DCM. Yield: 310 mg (99%); ¹H NMR (400 MHz, METHANOL-D₄) δ 1.45 (s, 1H), 1.50-1.64 (m, 4H), 1.68 (s, 9H), 1.70-1.85 (m, 4H), 1.96 (s, 1H), 2.01-2.12 (m, 2H), 2.27 (s, 1H), 3.24 (s, 1H), 3.40 (s, 1H), 3.62 (s, 1H), 3.87-3.95 (m, 1H), 4.19 (s, 1H), 4.57 (d, J=7.62 Hz, 2H), 7.61 (dd, J=8.59, 1.56 Hz, 1H), 7.78 (d, J=0.78 Hz, 1H), 7.99 (d, J=8.59 Hz, 1H); MS (ESI) (M+H)⁺ 434.2; Anal. Calcd for C₂₄H₃₃N₃O₂F₂+1.8 TFA+0.3 H₂O: C, 51.46; H, 5.54; N, 6.52. Found: C, 51.44; H, 5.37; N, 6.71.

### Example 17

### 2-tent-Butyl-5-{[4-(cyclopropylmethoxy)piperidin-1-yl]carbonyl}-1-[(4,4-difluorocycloheryl)methyl]-1H-benzimidazole

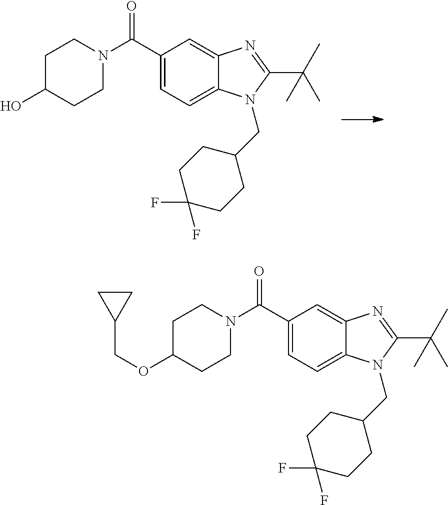

To a solution of NaH (0.011 g, 0.218 mmol) in 2 mL of DMF at 0° C. under nitrogen, a DMF solution (5 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.079 g, 0.182 mmol) was added dropwise. The solution was stirred at 0° C. under nitrogen for 30 min. (Cyclopropylmethyl)bromide (0.027 mL, 0.218 mmol) was added dropwise and the solution was stirred at it for 3 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 95 mg (87%); ¹H NMR (400 MHz, METHANOL-D₄) δ 0.16-0.22 (m, 2H), 0.47-0.54 (m, 2H), 0.98-1.07 (m, 1H), 1.50-1.63 (m, 4H), 1.68 (s, 11H), 1.71-1.85 (m, 4H), 1.95-2.11 (m, 3H), 2.27 (s, 1H), 3.31-3.37 (m, 2H), 3.51 (s, 1H), 3.59 (s, 1H), 3.63-3.70 (m, 1H), 4.08 (s, 1H), 4.57 (d, J=7.62 Hz, 2H), 7.62 (dd, J=8.59, 1.37 Hz, 1H), 7.78 (d, J=0.98 Hz, 1H), 8.00 (d, J=8.59 Hz, 1H); MS (ESI) (M+H)⁺ 488.3; Anal. Calcd for C₂₈H₃₉N₃O₂F₃+1.6 TFA+0.1 H₂O: C, 55.78; H, 6.12; N, 6.25. Found: C, 55.71; H, 6.07; N, 6.32.

### Example 18

### 2-tert-Butyl-5-{[4-(cyclobutylmethoxy)piperidin-1-yl]carbonyl}-1-[(4,4-difluorocyclohexyl)methyl]-1H-benzimidazole

To a solution of NaH (0.012 g, 0.288 mmol) in 2 mL of DMF at 0° C. under nitrogen, a DMF solution (5 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.050 g, 0.115 mmol) was added dropwise. The solution was stirred at 0° C. under nitrogen for 30 min. (Bromomethyl)cyclobutane (0.032 mL, 0288 mmol) was added dropwise and the solution was stirred at rt for 3 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 20 mg (29%); ¹H NMR (400 MHz, METHANOL-D₄) δ 1.50-1.64 (m, 4H), 1.67 (s, 9H), 1.69-1.80 (m, 7H), 1.84-1.98 (m, 3H), 2.00-2.09 (m, 5H), 2.26 (s, 1H), 2.49-2.58 (m, 1H), 3.46 (s, 2H), 3.56 (s, 1H), 3.58-3.65 (m, 2H), 4.03 (s, 1H), 4.56 (d, J=7.62 Hz, 2H), 7.60 (dd, J=8.69, 1.46 Hz, 1H), 7.77 (d, J=0.98 Hz, 1H), 7.98 (d, J=8.59 Hz, 1H); MS (ESI) (M+H)⁺502.3; Anal. Calcd for C₂₉H₄₁N₃O₂F₂+1.7 TFA+0.4 H₂O: C, 55.38; H, 6.24; N, 5.98. Found: C, 55.36; H, 6.21; N, 5.90.

### Example 19

### 2-tert-Butyl-1-[(4,4-difluorocyclohexyl)methyl]-5-[(4-propoxypiperidin-1-yl)carbonyl]-1H-benzimidazole

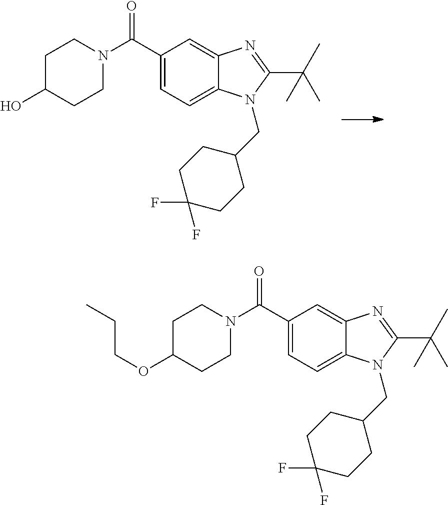

To a solution of NaH (0.012 g, 0.288 mmol) in 2 mL of DMF at 0° C. under nitrogen, a DMF solution (5 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.050 g, 0.115 mmol) was added dropwise. The solution was stirred at 0° C. under nitrogen for 30 min. Iodopropane (0.028 mL, 0.288 mmol) was added dropwise and the solution was stirred at rt for 3 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 40 mg (59%); ¹H NMR (400 MHz, METHANOL-D₄) δ 0.93 (t, J=7.42 Hz, 3H), 1.50-1.63 (m, 6H), 1.67 (s, 10H), 1.69-1.77 (m, 3H), 1.76-1.86 (m, 2H), 1.97 (s, 1H), 2.01-2.11 (m, 2H), 2.20-2.32 (m, 1H), 3.42-3.48 (m, 2H), 3.50-3.58 (m, 1H), 3.59-3.66 (m, 2H), 4.04 (s, 1H), 4.55 (d, J=7.62 Hz, 2H), 7.59 (dd, J=8.69, 1.46 Hz, 1H), 7.77 (d, J=0.98 Hz, 1H), 7.96 (d, J=8.59 Hz, 1H); MS (ESI) (M+H)⁺ 476.3.

### Example 20

### 2-tert-Butyl-1-[(4,4-difluorocyclohexyl)methyl]-5-[(4-isopropoxypiperidin-1-yl)carbonyl]-1H-benzimidazole

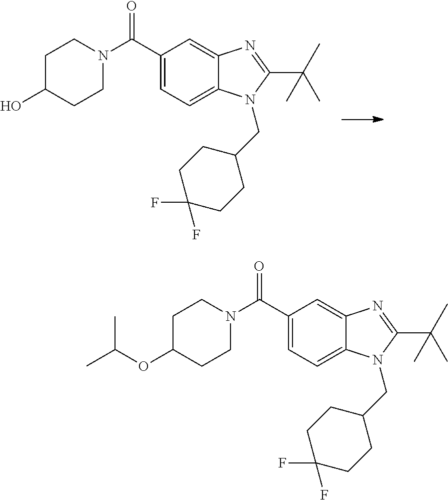

To a solution of NaH (0.010 g, 0.252 mmol) in 2 mL of DMF at 0° C. under nitrogen, a DMF solution (3 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.044 g, 0.101 mmol) was added dropwise. The solution was stirred at 0° C. under nitrogen for 30 min. 2-Iodopropane (0.026 mL, 0.252 mmol) was added dropwise and the solution was stirred at 75° C. for 24 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 15 mg (25%); ¹H NMR (400 MHz, METHANOL-D₄) δ 1.14 (d, J=5.66 Hz, 6H), 1.48 (s, 1H), 1.50-1.64 (m, 4H), 1.65-1.71 (m, 10H), 1.71-1.83 (m, 4H), 1.96 (s, 1H), 2.01-2.12 (m, 2H), 2.21-2.33 (m, 1H), 3.49 (s, 1H), 3.60 (s, 1H), 3.70-3.75 (m, 1H), 3.74-3.82 (m, 1H), 4.09 (s, 1H), 4.57 (d, J=7.62 Hz, 2H), 7.62 (dd, J=8.59, 1.56 Hz, 1H), 7.78 (d, J=0.78 Hz, 1H), 8.00 (d, J=8.59 Hz, 1H); MS (ESI) (M±H)⁺ 476.3.

### Example 21

### 2-tert-Butyl-1-[(4,4-difluorocyclohexyl)methyl]-5-{[4-(2,2-dimethylpropoxy)piperidin-1-yl]carbonyl}-1H-benzimidazole

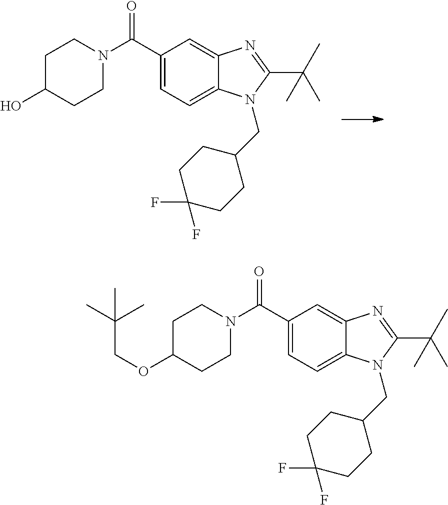

To a solution of NaH (0.017 g, 0.414 mmol) in 2 mL of DMF at 0° C. under nitrogen, a DMF solution (2 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.060 g, 0.138 mmol) was added dropwise. The solution was stirred at 0° C. under nitrogen for 30 min. 1-Bromo-2,2-dimethylpropane (0.035 mL, 0.276 mmol) was added dropwise and the solution was stirred at 100° C. overnight. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 45 mg (53%); ¹H NMR (400 MHz, METHANOL-D₄) δ 0.89-0.93 (m, 9H), 1.51-1.64 (m, 2H), 1.65-1.70 (m, 10H), 1.70-1.85 (m, 6H), 1.94 (s, 1H), 2.01-2.12 (m, 2H), 2.20-2.32 (m, 1H), 3.14 (d, J=8.59 Hz, 2H), 3.32 (s, 1H), 3.54-3.62 (m, 2H), 3.67 (s, 1H), 3.93 (s, 1H), 4.57 (d, J=7.62 Hz, 2H), 7.62 (dd, J=8.69, 1.46 Hz, 1H), 7.78 (d, J=0.98 Hz, 1H), 7.99 (d, J=8.59 Hz, 1H); MS (ESI) (M+H)⁺ 504.3.

### Example 22

### 5-{[4-(Benzyloxy)piperidin-1-yl]carbonyl}-2-tert-butyl-1-[(4,4-difluorocyclohexyl)methyl]-1H-benzimidazole

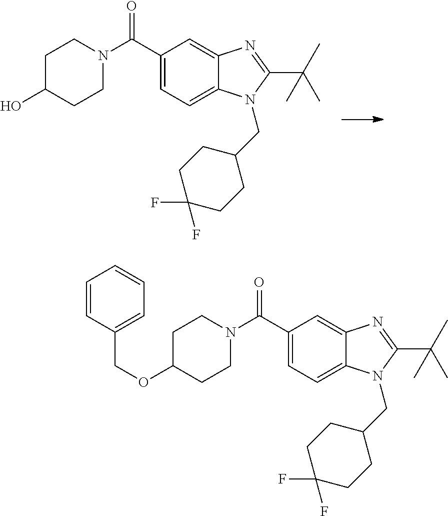

To a solution of NaH (0.014 g, 0.345 mmol) in 2 mL of DMF at 0° C. under nitrogen, a DMF solution (2 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.050 g, 0.115 mmol) was added dropwise. The solution was stirred at 0° C. under nitrogen for 30 min. Benzyl bromide (0.027 mL, 0.230 mmol) was added dropwise and the solution was stirred at rt for 2 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 50 mg (68%); ¹H NMR (400 MHz, METHANOL-D₄) δ 1.51-1.64 (m, 3H), 1.68 (s, 10H), 1.70-1.80 (m, 4H), 1.84 (s, 1H), 1.99-2.11 (m, 3H), 2.27 (s, 1H), 3.60 (s, 2H), 3.73-3.80 (m, 1H), 4.06 (s, 1H), 4.55-4.59 (m, 4H), 7.22-7.29 (m, 1H), 7.29-7.36 (m, 4H), 7.62 (dd, J=8.59, 1.37 Hz, 1H), 7.79 (d, J=0.78 Hz, 1H), 8.00 (d, J=8.59 Hz, 1H); MS (ESI) (M+H)⁺ 524.3.

### Example 23

### 2-tert-Butyl-1-[(4,4-difluorocyclohexyl)methyl]-5-[(4-isobutoxypiperidin-1-yl)carbonyl]-1H-benzimidazole

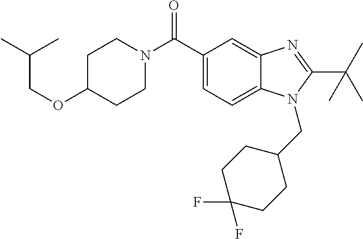

### Step A: 2-tert-Butyl-1-[(4,4-difluorocyclohexyl)methyl]-5-[(4-isobutoxypiperidin-1-yl)carbonyl]-1H-benzimidazole

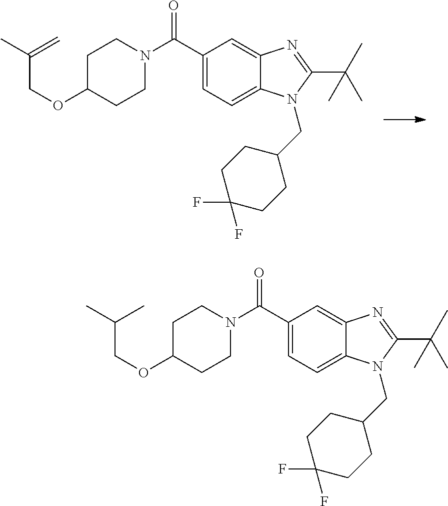

2-tert-Butyl-1-[(4,4-difluorocyclohexyl)methyl]-5-({4-[(2-methylprop-2-en-1-yl)oxy]piperidin-1-yl}carbonyl)-1H-benzimidazole (for preparation see the following Step B) (0.048 g, 0.0984 mmol) was shaken in 15 mL of EtOAc containing a catalytic amount of 10% Pd/C under H₂ atmosphere (45 psi) at it in a Parr hydrogenation apparatus for 12 h. The solution was filtered through a pad of celite and the solvent was evaporated. The product was purified by reversed-phase HPLC using 20-50% CH₃CN/H₂O and lyophilized to afford the title compound as the corresponding TFA salt. Yield: 49 mg (83%); ¹H NMR (400 MHz, METHANOL-D₄) δ 0.91 (d, J=6.64 Hz, 6H), 1.51-1.63 (m, 3H), 1.65-1.71 (m, 11H), 1.71-1.77 (m, 3H), 1.77-1.86 (m, 3H), 1.96 (s, 1H), 2.01-2.13 (m, 2H, 2.22-2.32 (m, 1H), 3.25 (t, J=5.66 Hz, 2H), 3.55-3.65 (m, 3H), 4.00 (s, 1H), 4.57 (d, J=7.42 Hz, 2H), 7.62 (dd, J=8.59, 1.56 Hz, 1H), 7.78 (dd, J=1.56, 0.59 Hz, 1H), 8.00 (d, J=8.59 Hz, 1H); MS (ESI) (M+H)⁺ 490.3.

### Step B: 2-tert-Butyl-1-[(4,4-difluorocyclohexyl)methyl]-5-({4-[(2-methylprop-2-en-1-yl)oxy]piperidin-1-yl}carbonyl)-1H-benzimidazole

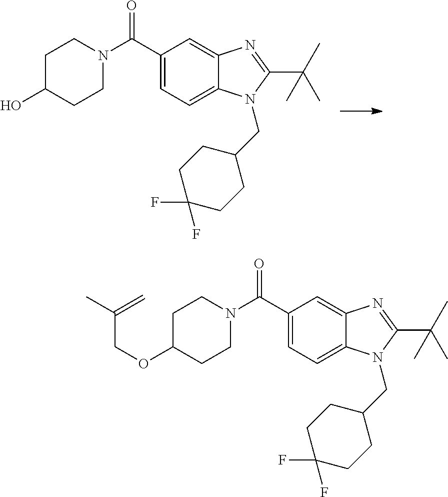

To a solution of NaH (0.017 g, 0.414 mmol) in 2 mL of DMF at 0° C. under nitrogen, a DMF solution (2 mL) of 1-({2-tert-butyl-1-[(4,4-difluorocyclohexyl)methyl]-1H-benzimidazol-5-yl}carbonyl)piperidin-4-ol (for preparation, see Example 16, Step B) (0.060 g, 0.138 mmol) was added dropwise. The solution was stirred at 0° C. under nitrogen for 30 min. 3-Bromo-2-methylpropen (0.028 mL, 0.276 mmol) was added dropwise and the solution was stirred at rt for 2 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified silica gel flash chromatography using 50% to 100% EtOAc/hexanes. Yield: 51 mg (76%); ¹H NMR (400 MHz, CHLOROFORM-D) δ 1.43-1.53 (m, 2H), 1.57 (s, 13H), 1.60-1.73 (m, 5H), 1.75 (s, 3H), 1.88 (s, 1H), 2.07-2.20 (m, 3H), 3.42 (s, 2H), 3.57-3.66 (n, 1H), 3.93 (s, 2H), 4.23 (d, J=7.42 Hz, 2H), 4.89 (s, 1H), 4.98 (s, 1H), 7.30-7.39 (m, 2H), 7.76 (s, 1H).

### Example 24

### 2-[1-({2-tert-Butyl-1-[(4,4-difluorocyclohexyl)methyl]-1H-benzimidazol-5-yl}carbonyl)azetidin-3-yl]-N-cyclopropylacetamide

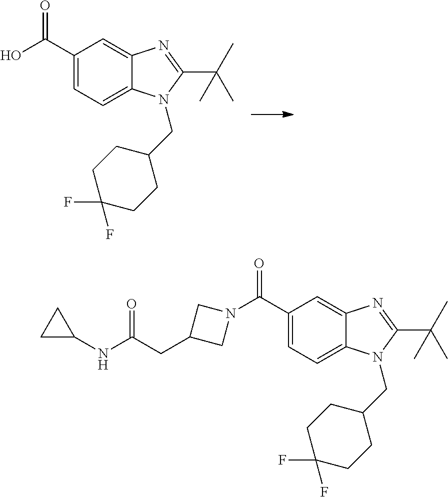

N-Boc-3-azetidine acetic acid (0.050 g, 0.232 mmol), HATU (0.105 g, 0.278 mmol) and cyclopropylaraine (0.020 mL, 0.278 mmol) were stirred in 3 mL of DMF containing DIPEA (0.061 mL, 0.348 mmol) at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The product was dissolved in 5 mL of 1M HCl/AcOH and stirred at room temperature for 2 h. The solvent was evaporated and the product was rinsed with ether and dried under vacuum. The product was dissolved in 3 mL of DMF and added to a mixture of 2-tert-butyl-1-[(4,4-difluorocyclohexyl)methyl]-1H-benzimidazole-5-carboxylic acid (for preparation see Example 1) (0.030 g, 0.0856 mmol), DIPEA (22 μL, 0.128 mmol) and HATU (0.039 g, 0.102 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 17 mg (33%). ¹H NMR (400 MHz, METHANOL-D₄) δ 0.40-0.47 (m, 2H), 0.65-0.72 (m, 2H), 1.50-1.62 (m, 2H), 1.68 (s, 9H), 1.69-1.82 (m, 4H), 2.00-2.10 (m, 3H), 2.26 (s, 1H), 2.51 (dd, J=7.71, 3.03 Hz, 2H), 2.57-2.64 (m, 1H), 2.99-3.08 (m, 1H), 3.90 (dd, J=10.35, 6.05 Hz, 0.5H), 4.14 (dd, J=8.69, 5.96 Hz, 0.5H), 4.33 (t, J=9.67 Hz, 0.5H), 4.51 (t, J=8.79 Hz, 0.5H), 4.56 (d, J=7.42 Hz, 2H), 7.83 (dd, J=8.69, 1.46 Hz, 1H), 7.96-8.01 (m, 2H); MS (ESI) (M+H)⁺ 487.0.

### Example 25

### N-[1-({2-tert-Butyl-1-[(4,4-difluorocyclohexyl)methyl]-1H-benzimidazol-5-yl}carbonyl)piperidin-4-yl]cyclopropanecarboxamide

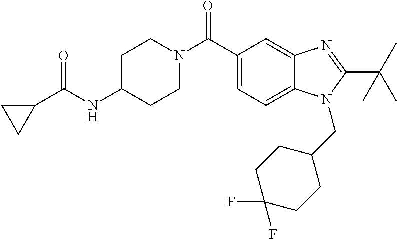

### Step A: N-[1-({2-tert-Butyl-[4(4,4-difluorocyclohexyl)methyl]-1H-benzimidazol-5-yl}carbonyl)piperidin-4-yl]cyclopropanecarboxamide

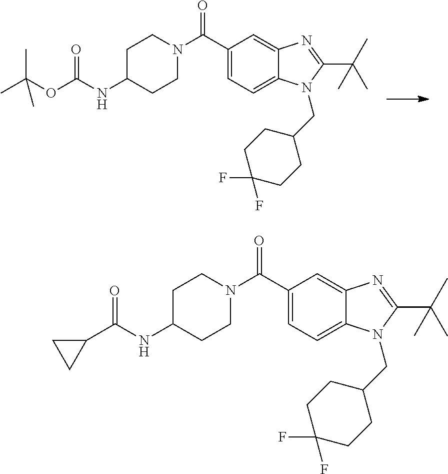

tert-Butyl[1-({2-tert-butyl-1-[(4,4-difluorocyclohexyl)methyl]-1H-benzimidazol-5-yl}carbonyl)piperidin-4-yl]carbamate (for preparation see the following step B) (0.065 g, 0.122 mmol) was stirred in 5 mL of 1M HCl/AcOH at room temperature for 1 h. The solvent was evaporated. CH₂Cl₂ was added to the resulting residue and the organic layer was washed once with a saturated aqueous NaHCO₃ solution, once with brine and dried over anhydrous Na₂SO₄. was removed under reduced pressure. The product was dissolved in 3 mL of CH₂Cl₂ containing triethylamine (0.025 mL, 0.183 mmol) and cyclopropanecarbonyl chloride (0.015 mL, 0.159 mmol) was added. The solution was stirred at room temperature for 1 h. The solution was washed once with a saturated aqueous NaHCO₃ solution, once with brine and dried over anhydrous Na₂SO₄. CH₂Cl₂ was removed under reduced pressure: The resulting residue was purified by reversed-phase HPLC using 20-50% CH₃CN/H₂O and lyophilized to afford the title compound as the corresponding TFA salt. Yield: 50 mg (67%). ¹H NMR (400 MHz, METHANOL-D₄) δ 0.73 (m, 2H), 0.81 (m, 2H), 1.40 (s, 1H), 1.49-1.64 (m, 3H), 1.68 (s, 9H), 1.70-1.80 (m, 4H), 1.79-1.92 (m, 1H), 2.00-2.13 (m, 3H), 2.21-2.32 (m, 1H), 3.11 (s, 1H), 3.24 (s, 1H), 3.67 (s, 1H), 3.89-3.98 (m, 1H), 4.57 (d, J=7.62 Hz, 2H), 7.62 (dd, J=8.59, 1.37 Hz, 1H), 7.79 (d, J=1.17 Hz, 1H), 8.01 (d, J=8.59 Hz, 1H); MS (ESI) (M+H)⁺ 501.3.

### Step B: tert-Butyl[1-({2-tert-butyl-1-[(4,4-difluorocyclohexyl)methyl]-1H-benzimidazol-5-yl}carbonyl)piperidin-4-yl]carbamate

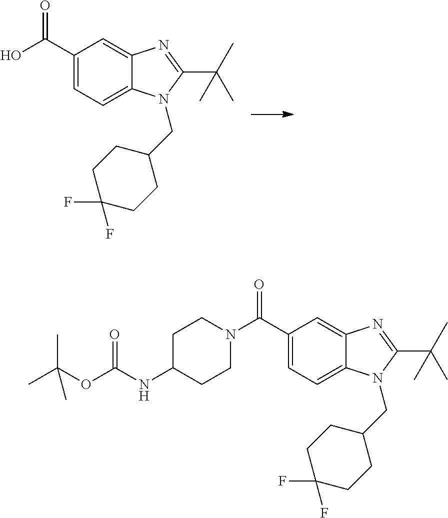

To a mixture of 2-tert-butyl-1-[(4,4-difluorocyclohexyl)methyl]-1H-benzimidazole-5-carboxylic acid (for preparation see Example 1) (0.050 g, 0.143 mmol), DIPEA (37 μL, 0.215 mmol) and DMF (2 mL) were added HATU (0.065 g, 0.172 mmol) and 4-(N-Boc-amino)-piperidine (0.035 g, 0.172 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by column chromatography on silica gel using 100% ethyl acetate. Yield: 68 mg (89%). ¹H NMR (400 MHz, CHLOROFORM-D) δ 1.34-1.42 (m, 2H), 1.45 (s, 9H), 1.46-1.54 (m, 3H), 1.57 (s, 9H), 1.60-1.66 (m, 2H), 1.66-1.74 (m, 4H), 1.98 (s, 2H), 2.08-2.19 (m, 3H), 3.06 (s, 2H), 4.23 (d, J=7.42 Hz, 2H), 4.44-4.51 (m, 1H), 7.33-7.35 (m, 2H), 7.73-7.75 (m, 1H).

### Example 26

### 1-({2-tert-Butyl-1-[(4,4-difluorocyclohexyl)methyl]-1H-benzimidazol-5-yl}carbonyl)-N-(cyclopropylmethyl)piperidin-4-amine

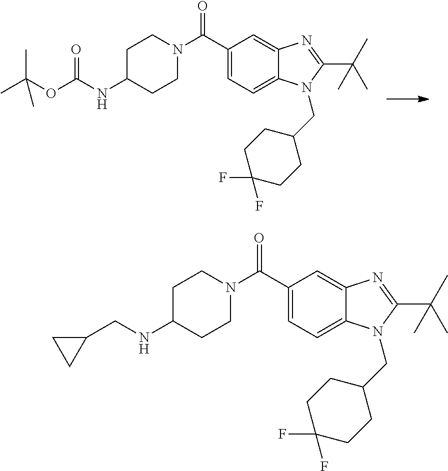

tert-Butyl[1-({2-tert-butyl-1-[(4,4-difluorocyclohexyl)methyl]-1H-benzimidazol-5-yl}carbonyl)piperidin-4-yl]carbamate (for preparation see Example 25, Step B) (0.076 g, 0.143 mmol) was dissolved in 5 mL of DMF at 0° C. under nitrogen. Nail (0.022 g, 0.572 mmol) was added and the solution was stirred at 0° C. for 10 min. (Cyclopropylmethyl)bromide (0.021 mL, 0.215 mmol) was added and the solution was stirred at rt for 2 h. The reaction was quenched with a few drops of saturated aqueous NaHCO₃ solution and the solvent was evaporated. CH₂Cl₂ was added to the resulting residue and the organic layer was washed once with a saturated aqueous NaHCO₃ solution, to once with brine and dried over anhydrous Na₂SO₄. CH₂Cl₂ was removed under reduced pressure. The product was stirred in 3 mL of 1M HCl/AcOH at room temperature for 1 h. The solvent was evaporated. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting product was purified by reversed-phase HPLC using 20-50% CH₃CN/H₂O and lyophilized to afford the title compound as the corresponding TFA salt. Yield: 58 mg (68%). ¹H NMR (400 MHz, METHANOL-D₄) δ 0.36-0.43 (m, 2H), 0.66-0.75 (m, 2H), 1.02-1.12 (m, 1H), 1.50-1.64 (m, 4H), 1.68 (s, 9H), 1.70-1.85 (m, 4H), 2.01-2.13 (m, 3H), 2.26 (s, 2H), 2.95 (d, J=7.42 Hz, 2H), 3.23 (s, 1H), 3.37-3.50 (m, 1H), 3.82 (s, 1H), 4.57 (d, J=7.62 Hz, 2H), 4.77 (s, 1H), 7.61 (dd, J=8.59, 1.37 Hz, 1H), 7.81 (d, J=0.98 Hz, 1H,) 8.00 (d, J=8.79 Hz, 1H); MS (ESI) (M+H)⁺ 487.3.

### Example 27

### 4-({2-tert-Butyl-1-[(4,4-difluorocyclohexyl)methyl]-1H-benzimidazol-5-yl}carbonyl)-N-cyclopropylpiperazine-1-carboxamide

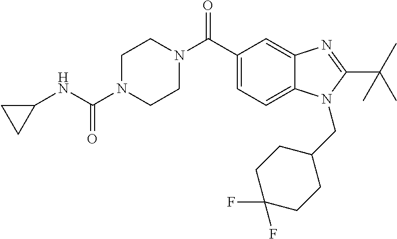

### Step A: 4-({2-tert-Butyl-1-[(4,4-difluorocyclohexyl)methyl]-1H-benzimidazol-5-yl}carbonyl)-N-cyclopropylpiperazine-1-carboxamide

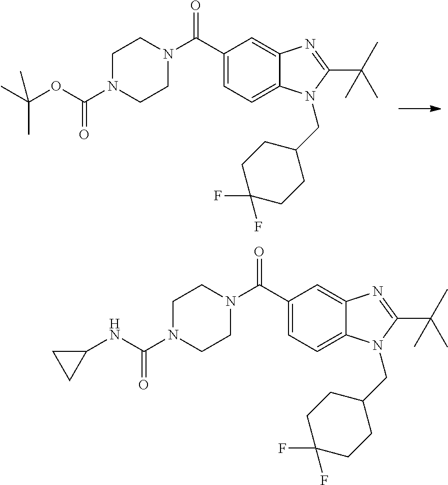

tert-Butyl 4-({2-tert-butyl-1-[(4,4-difluorocyclohexyl)methyl]-1H-benzimidazol-5-yl}carbonyl)piperazine-1-carboxylate (for preparation see following step B) (105 mg, 0.202 mmol) was stirred in 2 mL of 1M HCl/AcOH at room temperature for 1 h. The solvent was evaporated. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. To a solution of triphosgene (0.060 g, 0.202 mmol) in 3 mL of DCM was added a solution of cyclopropylamine (0.042 mL, 0.606 mmol) and DIPEA (0.175 mL, 1.01 mmol) in 2 mL of DCM at 0° C. The amine in 2 mL of DCM was then added and the solution was stirred at room temperature for 20 min. The solution mixture was washed once with a saturated aqueous NaHCO₃ solution, once with brine and dried over anhydrous Na₂SO₄. CH₂Cl₂ was removed under reduced pressure. The resulting product was purified by reversed-phase HPLC using 20-50% CH₃CN/H₂O and lyophilized to afford the title compound as the corresponding TFA salt. Yield: 100 mg (80%). ¹H NMR (400 MHz, METHANOL-D₄) δ 0.41-0.46 (m, 2H), 0.62-0.68 (m, 2H), 0.70-0.75 (m, 1H), 0.81-0.87 (m, 1H), 1.51-1.62 (m, 2H), 1.67 (s, 9H), 1.70-1.82 (m, 4H), 2.01-2.11 (m, 2H), 2.21-2.30 (m, 1H), 2.49-2.56 (m, 1H), 2.57-2.63 (m, 1H), 3.36-3.51 (m, 4H), 3.71-3.80 (m, 1H), 4.56 (d, J=7.42 Hz, 2H), 7.62 (dd, J=8.59, 1.37 Hz, 1H), 7.80 (d, J=0.78 Hz, 1H), 7.98 (d, J=8.59 Hz, 1H); MS (ESI) (M+H)⁺ 502.0.

### Step B: tert-Butyl 4-({2-tert-butyl-1-[(4,4-difluorocyclohexyl)methyl]-1H-benzimidazol-5-yl}carbonyl)piperazine-1-carboxylate

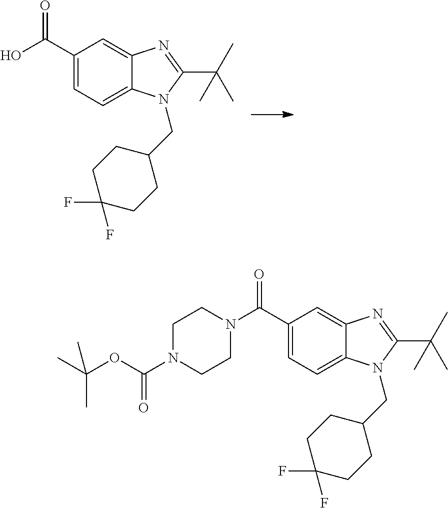

To a mixture of 2-tert-butyl-1-[(4,4-difluorocyclohexyl)methyl]-1H-benzimidazole-5-carboxylic acid (for preparation see Example 1) (0.075 g, 0.214 mmol), DIPEA (56 μL, 0.321 mmol) and DMF (3 mL) were added HATU (0.098 g, 0.257 mmol) and 1-Boc-piperazine (0.049 g, 0.257 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by column chromatography on silica gel using 100% ethyl acetate. Yield: 100 mg (99%). ¹H NMR (400 MHz, CHLOROFORM-D) δ 1.41-1.48 (m, 11H), 1.56 (s, 9H), 1.59-1.65 (m, 2H), 1.65-1.73 (m, 4H), 2.08-2.18 (m, 3H), 3.45 (s, 4H), 3.62 (s, 2H), 4.23 (d, J=7.42 Hz, 2H), 7.34-7.36 (m, 2H), 7.75 (s, 1H).

### Example 28

### 2-tert-Butyl-5-{[4-(cyclopropylcarbonyl)piperazin-1-yl]carbonyl}-1-[(4,4-difluorocyclohexyl)methyl]-1H-benzimidazole

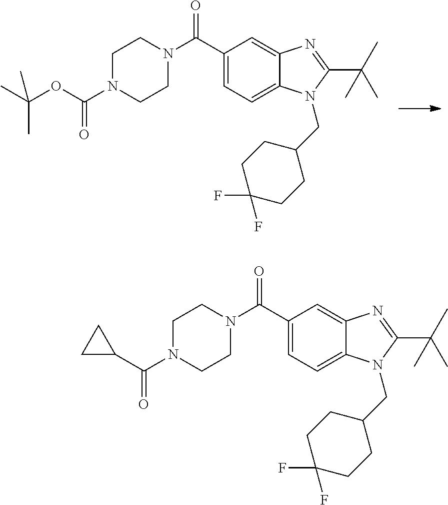

tert-Butyl 4-({2-tert-butyl-1-[(4,4-difluorocyclohexyl)methyl]-1H-benzimidazol-5-yl}carbonyl)piperazine-1-carboxylate (for preparation see Step B, Example 27) (0.074 g, 0.143 mmol) was stirred in 3 mL of 1M HCl/AcOH at room temperature for 1 h. The solvent was evaporated. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The product was dissolved in 5 mL of CH₂Cl₂ containing triethylamine (0.030 mL, 0.215 mmol) and cyclopropanecarbonyl chloride (0.017 mL, 0.186 mmol) was added. The solution was stirred at room temperature for 1 h. The solution mixture was washed once with a saturated aqueous NaHCO₃ solution, once with brine and dried over anhydrous Na₂SO₄. CH₂Cl₂ was removed under reduced pressure. The resulting product was purified by reversed-phase HPLC using 20-50% CH₃CN/H₂O and lyophilized to afford the title compound as the corresponding TFA salt. Yield: 58 mg (68%). ¹H NMR (400 MHz, METHANOL-D₄) δ 0.80-0.85 (m, 2H), 0.85-0.91 (m, 2H), 1.50-1.63 (m, 2H), 1.69 (s, 9H), 1.70-1.84 (m, 4H), 1.99-2.12 (m, 3H), 2.20-2.34 (m, 1H), 3.38-3.65 (m, 4H), 3.68-3.98 (m, 4H), 4.58 (d, J=7.62 Hz, 2H), 7.68 (dd, J=8.59, 1.37 Hz, 1H), 7.84 (s, 1H), 8.03 (d, J=8.79 Hz, 1H); MS (ESI) (M+H)⁺ 487.3.

### Example 29

### 2-[1-({2-tert-Butyl-1-[(4,4-difluorocyclohexyl)methyl]-1H-benzimidazol-5-yl}carbonyl)piperidin-4-yl]-N-cyclopropylacetamide

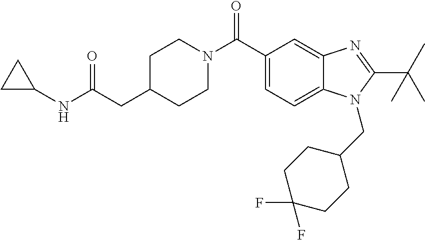

### Step A: 2-[1-({2-tert-Butyl-1-[(4,4-difluorocyclohexyl)methyl]-1H-benzimidazol-5-yl}carbonyl)piperidin-4-yl]-N-cyclopropylacetamide

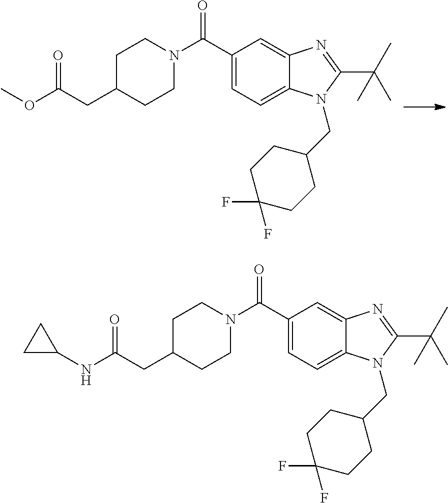

Methyl[1-({2-tert-butyl-1-[(4,4-difluorocyclohexyl)methyl]-1H-benzimidazol-5-yl}carbonyl)piperidin-4-yl]acetate (for preparation, see following Steps B and C) (0.049 g, 0.100 mmol) was stirred in 5 mL of dioxane containing 1 mL of 1M LiOH at 75° C. for 2 h. The solvent was evaporated. The residue was acidified to pH=5-6 with aqueous 5% KHSO₄ solution and extracted with ether (2×) and CH₂Cl₂ (2×). The organic phases were combined, washed with brine, dried over anhydrous Na₂SO₄ and concentrated. The product was dissolved in 3 mL of DMF containing DIPEA (0.026 mL, 0.150 mmol) and cyclopropylarnine (0.008 mL, 0.120 mmol), and HATU (0.046 g, 0.120 mmol) was added. The solution was stirred at it for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting product was purified by reversed-phase HPLC using 20-50% CH₃CN/H₂O and lyophilized to afford the title compound as the corresponding TFA salt. Yield: 46 mg (74%); ¹H NMR (400 MHz, METHANOL-D₄) δ 0.41-0.47 (m, 2H), 0.66-0.73 (m, 2H), 1.14-1.32 (m, 1H), 1.51-1.64(m,2H), 1.67-1.70 (m, 11H), 1.72-1.87 (m, 5H), 2.00-2.13 (m, 5H), 2.21-2.31 (m, 1H,) 2.58-2.67 (m, 1H), 2.91 (t, J=13.77 Hz, 1H), 3.14 (t, J=13.57 Hz, 1H), 3.62-3.72 (m, 1H), 4.57 (d, J=7.62 Hz, 2H), 4.61 (dd, 1H,) 7.61 (dd, J=8.59, 1.56 Hz, 1H), 7.76 (d, J=0.98 Hz, 1H), 8.00 (d, J=8.79 Hz, 1H); MS (ESI) (M+H)⁺ 515.3; Anal. Calcd for C₂₉H₄₀N₄O₂F₂+2.1 TFA+2.4 H₂O: C, 50.01; H, 5.93; N, 7.03. Found: C, 49.97; H, 5.83; N, 7.39.

### Step B: Methyl piperidin-4-ylacetate hydrochloride

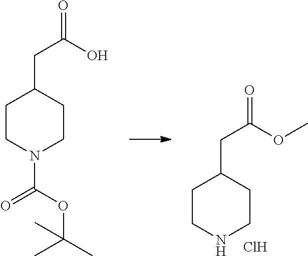

Boc-(4-Carboxymethyl)-piperidine (0.100 g, 0.411 mmol) was dissolved in 3 mL of MeOH at 0° C. 2M TMSCHN₂/hexanes was added dropwise at 0° C. until a light yellow color persisted. The solution was let to stir at rt for 30 minutes. The solvent was evaporated. The residue was dissolved in EtOAc and washed with a 5% aqueous KHSO₄ solution, saturated aqueous NaHCO₃ solution, brine and dried over anhydrous Na₂SO₄. The solvent was evaporated. The residue was dissolved in 5 mL of 1M HCl/AcOH and the solution was stirred at rt for 1 h. The solvent was evaporated. The residue was crashed in ether, filtered and dried under vacuum. Yield: 70 mg (89%); ¹H NMR (400 MHz, METHANOL-D₄) δ 1.37-1.52 (m, 2H), 1.95 (d, J=13.87 Hz, 2H), 2.00-2.14 (m, 1H), 2.34 (d, J=7.03 Hz, 2H), 2.98 (t, J=12.89 Hz, 2H), 3.35 (d, J=12.69 Hz, 2H), 3.65 (s, 3H).

### Step C: Methyl[1-({2-tert-butyl-1-[(4,4-difluorocyclohexyl)methyl]-1H-benzimidazol-5-yl}carbonyl)piperidin-4-yl]acetate

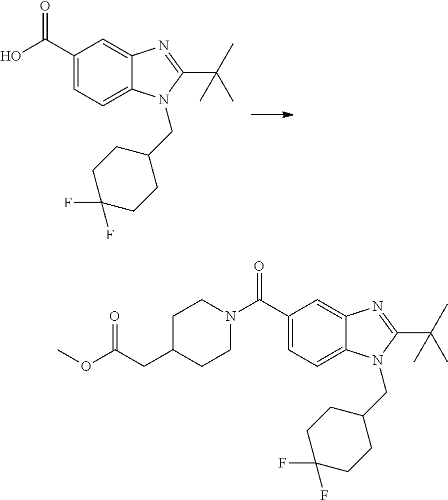

To a mixture of 2-tert-butyl-1-[(4,4-difluorocyclohexyl)methyl]-1H-benzimidazole-5-carboxylic acid (for preparation see Example 1) (0.045 g, 0.128 mmol), DIPEA (0.056 mL, 0.320 mmol) and DMF (3 mL) were added HATU (0.059 g, 0.154 mmol) and methyl piperidin-4-ylacetate hydrochloride (0.030 g, 0.154 mmol). The mixture was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by column chromatography on silica gel using 100% EtOAc. Yield: 52 mg (84%); ¹H NMR (400 MHz, CHLOROFORM-D) δ 1.21-1.36 (m, 2H), 1.42-1.55 (m, 2H), 1.57 (s, 9H), 1.63-1.84 (m, 7H), 2.06-2.19 (m, 4H), 2.29 (d, J=6.84 Hz, 2H), 2.94 (s, 1H), 3.68 (s, 3H), 4.23 (d, J=7.42 Hz, 2H), 7.32-7.36 (m, 2H), 7.74 (s, 1H).

### Example 30

### 2-tert-Butyl-5-{[3-(cyclopropylmethoxy)azetidin-1-yl]carbonyl}-1-[(4,4-difluorocyclohexyl)methyl]-1H-benzimidazole

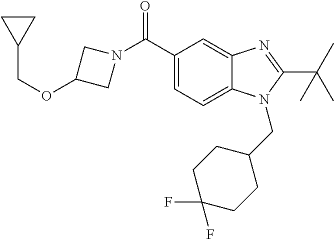

### Step A: 2-tert-Butyl-5-{[3-(cyclopropylmethoxy)azetidin-1-yl]carbonyl}-1-[(4,4-difluorocyclohexyl)methyl]-1H-benzimidazole

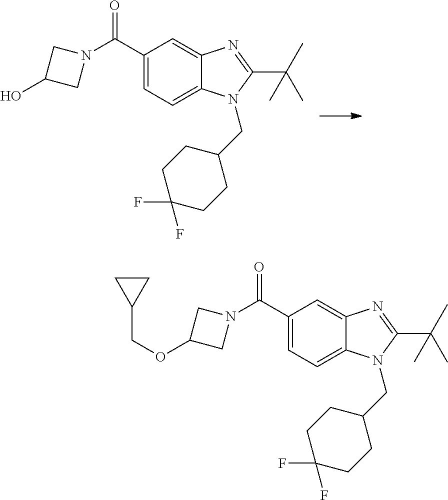

To a solution of NaH (0.010 g, 0.266 mmol) in 2 mL of DMF at 0° C. under nitrogen, a DMF solution (2 mL) of 1-({2-tert-butyl-1-[(4,4-difluorocyclohexyl)methyl]-1H-benzimidazol-5-yl}carbonyl)azetidin-3-ol (for preparation, see following Step B) (0.054 g, 0.133 mmol) was added dropwise. The solution was stirred at 0° C. under nitrogen for 30 min. (Cyclopropylmethyl)bromide (0.026 mL, 0.266 mmol) was added dropwise and the solution was stirred at it for 3 h. The reaction was quenched at 0° C. by addition of aqueous saturated NaHCO₃ solution and the solvent was concentrated. The residue was dissolved in EtOAc and washed with saturated aqueous NaHCO₃ solution, brine and dried over anhydrous Na₂SO₄. The product was purified by reversed-phase HPLC using 20-50% CH₃CN/H₂O and lyophilized to afford the title compound as the corresponding TFA salt. Yield: 60 mg (79%); ¹H NMR (400 MHz, METHANOL-D₄) δ 0.18-0.23 (m, 2H), 0.49-0.55 (m, 2H), 0.98-1.06 (m, 1H), 1.51-1.63 (m, 2H), 1.68 (s, 9H), 1.69-1.76 (m, 4H), 1.76-1.84 (m, 1H), 2.01-2.11 (m, 2H), 2.20-2.30 (m, 1H), 3.31 (d, J=1.76 Hz, 1H), 4.02-4.08 (m, 1H), 4.22-4.27 (m, 1H), 4.38-4.45 (m, 2H), 4.51-4.54 (m, 1H), 4.56 (d, J=7.62 Hz, 2H), 7.84 (dd, J=8.69, 1.46 Hz, 1H), 7.98 (d, J=8.79 Hz, 1H), 8.01 (d, J=1.17 Hz, 1H); MS (ESI) (M+H)⁺460.3; Anal. Calcd for C₂₆H₃₅N₃O₂F₂+1.7 TFA+0.3 H₂O: C, 53.60; H, 5.71; N, 6.38. Found: C, 53.60; H, 5.69; N, 6.16.

### Step B: 1-({2-tert-Butyl-1-[(4,4-difluorocyclohexyl)methyl]-1H-benzimidazol-5-yl}carbonyl)azetidin-3-ol

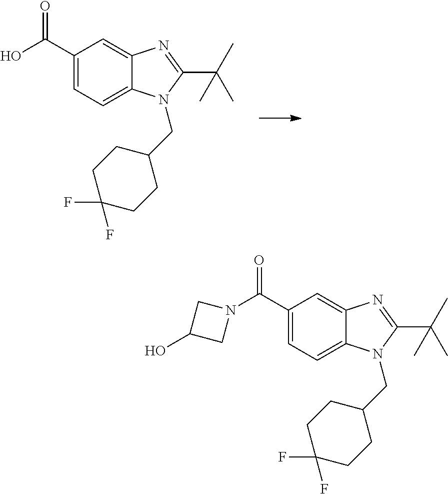

To a mixture of 2-tert-butyl-1-[(4,4-difluorocyclohexyl)methyl]-1H-benzimidazole-5-carboxylic acid (for preparation see Example 1) (0.100 g, 0.285 mmol), DIPEA (0.125 mL, 0.712 mmol) and DMF (5 mL) were added HATU (0.130 g, 0.342 mmol) and 3-hydroxyazetidine hydrochloride (0.037 g, 0.342 mmol). The mixture was stirred at rt for 2 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting product was purified by silica gel flash chromatography using EtOAc as eluent. Yield: 110 mg (96%); ¹H NMR (400 MHz, CHLOROFORM-D) δ 1.43-1.55 (m, 2H), 1.57 (s, 9H), 1.60-1.64 (m, 1H), 1.64-1.75 (m, 3H), 2.08-2.19 (m, 3H), 4.07-4.17 (m, 1H), 4.24 (d, J=7.62 Hz, 2H), 4.29 (s, 1H), 4.54 (s, 2H), 4.70-4.78 (m, 1H), 7.36 (d, J=8.40 Hz, 1H), 7.69 (dd, J=8.50, 1.66 Hz, 1H), 7.93 (d, J=1.17 Hz, 1H).

### Example 31

### 1-{[2-tert-Butyl-1-(cyclohexyhnethyl)-1H-benzimidazol-5-yl]carbonyl}-N-cyclopropylpiperidine-4-carboxamide

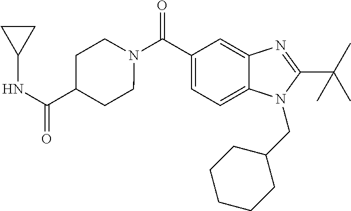

### Step A: 1-{[2-tert-Butyl-1-(cyclohexylmethyl)-1H-benzimidazol-5-yl]carbonyl}-N-cyclopropylpiperidine-4-carboxamide

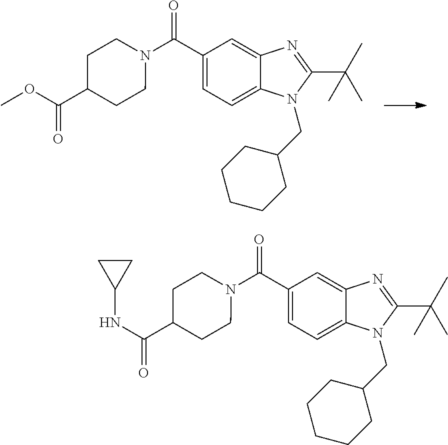

Methyl 1-{[2-tert-butyl-1-(cyclohexylmethyl)-1H-benzimidazol-5-yl]carbonyl}piperidine-4-carboxylate (for preparation see the following steps B to F) (0.068 g, 0.155 mmol) was heated in 6 mL of a 5:1 mixture of dioxane: 1M LiOH at 75° C. for 3 h. The solvent was evaporated. The residue was dissolved in water and was acidified to pH 5-6 with a 5% aqueous KHSO₄ solution. The mixture was extracted twice with Et₂O. The organic layer was washed once with brine and dried over anhydrous Na₂SO₄. Et₂O was removed at reduced pressure. The product was dissolved in 5 mL of DMF containing DIPEA (0.040 mL, 0.233 mmol), cyclopropylamine (0.013 mL, 0.186 mmol) and HATU (0.070 g, 0.186 mmol) and the solution was stirred at room temperature for 1 h. The solvent was removed under reduced pressure. CH₂Cl₂ was added to the resulting residue and the organic layer was washed once with a saturated aqueous NaHCO₃ solution, once with brine and dried over anhydrous Na₂SO₄. CH₂Cl₂ was removed under reduced pressure. The resulting residue was purified by reversed-phase HPLC using 20-50% CH₃CN/H₂O and lyophilized to afford the title compound as the corresponding TFA salt. Yield: 70 mg (78%). ¹H NMR (400 MHz, METHANOL-D₄) δ 0.42-0.48 (m, 2H), 0.66-0.74 (m, 2H), 1.19-1.29 (m, 5H), 1.61-1.66 (m, 3H), 1.66-1.71 (m, 11H), 1.77 (s, 3H), 1.86 (s, 1H), 2.13 (s, 1H), 2.40-2.49 (m, 1H), 2.59-2.67 (m, 1H), 2.94 (s, 1H), 3.15 (s, 1H), 3.70 (s, 1H), 4.49 (d, J=7.62 Hz, 2H), 4.66 (s, 1H), 7.62 (dd, J=8.69, 1.46 Hz, 1H), 7.79 (d, J=1.17 Hz, 1H), 8.00 (d, J=8.59 Hz, 1H); MS (ESI) (M+H)⁺ 465.3.

### Step B: Methyl 4-[(cyclohexylmethyl)amino]-3-nitrobenzoate

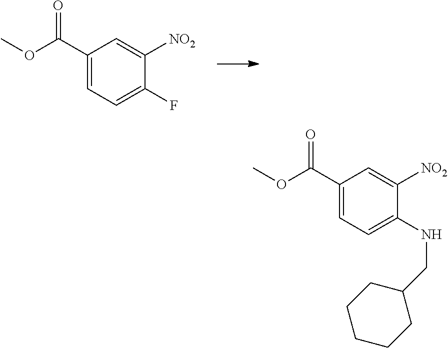

Following the same procedure as in Example 1, step B, using methyl 4-fluoro-3-nitrobenzoate (225 mg, 1.13 mmol) and cyclohexylmethylamine (0.175 mL, 1.36 mmol). The product was directly used for next step after the regular washings. Yield: 329 mg (99%). ¹H NMR (400 MHz, CHLOROFORM-D) δ 1.06 (m, 2H), 1.26 (m, 3H), 1.72 (m, 3H), 1.72 (m, 2H), 1.86 (m, 2H), 3.20 (dd, J=6.64, 5.47 Hz, 2H), 3.90 (s, 3H), 6.86 (d, J=8.98Hz, 1H), 8.04 (ddd, J=9.03, 2.10, 0.78 Hz, 1H), 8.47 (s, 1H), 8.89 (d, J=1.95 Hz, 1H).

### Step C: Methyl 3-amino-4-[(cyclohexylmethyl)amino]benzoate

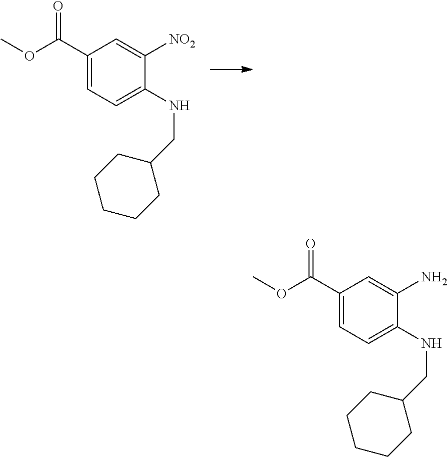

Same procedure used as for Example 1, step C using methyl 4-[(cyclohexylmethyl)amino]-3-nitrobenzoate (325 mg, 1.11 mmol). The solution was filtered through Celite and used directly for next step. Yield: 285 mg (98%). MS (ESI) (M+H)⁺ 263.0.

### Step D: Methyl 2-tert-butyl-1-(cyclohexylmethyl)-1H-benzimidazole-5-carboxylate

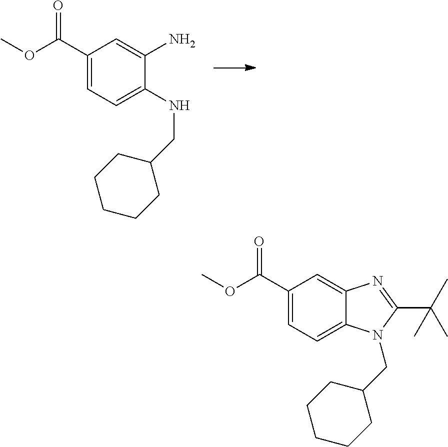

Methyl 3-amino-4-[(cyclohexylmethyl)amino]benzoate (285 mg, 1.09 mmol) was dissolved in 10 mL of DCM containing DMAP (33 mg, 0.272 mmol). Trimethylacetyl chloride (0.145 mL, 1.20 mmol) was added drop wise and the solution was stirred at room temperature for 2 h. The solvent was concentrated. The residue was dissolved in 15 mL of glacial AcOH and stirred at 100° C. for 24 h. The solvent was concentrated. The residue was dissolved in EtOAc and the solution was washed with saturated NaHCO₃ solution, brine and dried over anhydrous MgSO₄. The product was purified by flash chromatography using 7:3/hexanes:EtOAc. Yield: 170 mg (47%). ¹H NMR (400 MHz, CHLOROFORM-D) δ 1.10 (m, 2H), 1.16 (m, 2H), 1.57 (s, 9H), 1.62 (m, 3H), 1.69 (m, 1H), 1.73 (m, 2H), 2.03 (m, 1H), 3.93 (s, 3H), 4.15 (d, J=7.62 Hz, 2H), 7.34 (d, J=8.59 Hz, 1H), 7.94 (dd, J=8.59, 1.56 Hz, 1H), 8.47 (d, J=0.98 Hz, 1H).

### Step E: 2-tert-Butyl-1-(cyclohexylmethyl)-1H-benzimidazole-5-carboxylic acid

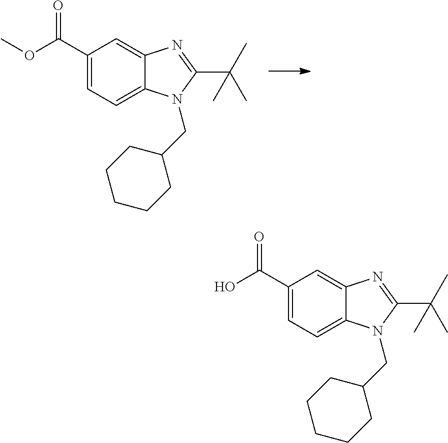

Methyl 2-tert-butyl-1-(cyclohexylmethyl)-1H-benzimidazole-5-carboxylate (165 mg, 0.502 mmol) was dissolved in 10 mL of EtOH containing 2 mL of 1M LiOH. The solution was refluxed for 3 h. The solution was cooled to room temperature and concentrated. The solution was neutralized with 1M HCl and extracted with CH₂Cl₂ and EtOAc. The organic phases were washed with brine and dried over anhydrous MgSO₄. The organic phases were combined and concentrated. Yield: 140 mg (87%). MS (ESI) (M+H)⁺ 315.0.

### Step F: Methyl 1-{[2-tert-butyl-1-(cyclohexylmethyl)-1H-benzimidazol-5-yl]carbonyl}piperidine-4-carboxylate

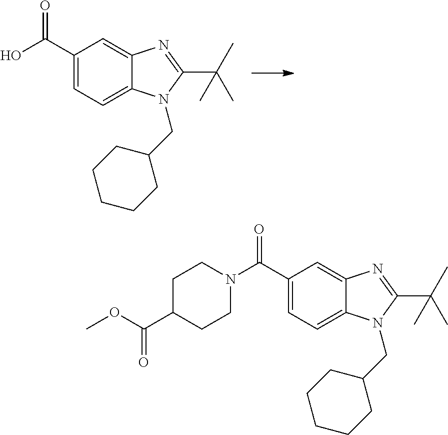

2-tert-Butyl-1-(cyclohexylmethyl)-1H-benzimidazole-5-carboxylic acid (0.075 g, 0.238 mmol), DIPEA (0.080 mL, 0.476 mmol) and DMF (5 mL) were added HATU (0.110 g, 0.286 mmol) and methyl isonipecotate (0.039 mL, 0.286 mmol). The mixture was stirred at room temperature for 1 h. The solvent was removed under reduced pressure. CH₂Cl₂ was added to the resulting residue and the organic layer was washed once with saturated aqueous NaHCO₃ solution, brine and dried over anhydrous Na₂SO₄. CH₂Cl₂ was removed under reduced pressure. The resulting residue was purified by column chromatography on silica gel using 100% ethyl acetate. Yield: 71 mg (68%). ¹H NMR (400 MHz, CHLOROFORM-D) δ 1.05-1.22 (m, 5H), 1.56 (s, 9H), 1.59-1.67 (m, 4H), 1.67-1.81 (m, 5H), 1.93 (s, 2H), 2.00-2.08 (m, 1H), 2.54-2.63 (m, 1H), 3.01-3.12 (m, 2H), 3.71 (s, 3H), 4.14 (d, J=7.62 Hz, 2H), 7.31-7.35 (m, 1H), 7.35-7.38 (m, 1H), 7.74 (s, 1H).

1. A compound of formula I, a pharmaceutically acceptable salt thereof, a diastereomer, an enantiomer, or a mixture thereof: 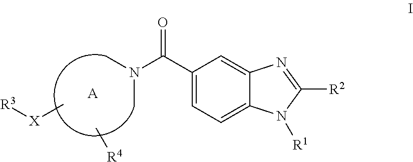
wherein:
R¹ is selected from C₁₋₁₀alkyl, C₂₋₁₀alkenyl, C₁₋₁₀alkoxy, C₆₋₁₀aryl-C₁₋₆alkyl, C₆₋₁₀aryl-C(═O)-C₁₋₆alkyl, C₃₋₁₀cycloalkyl-C₁₋₆alkyl, C₄₋₈cycloalkenyl-C₁₋₆alkyl, C₃₋₆heterocyclyl-C₁₋₆alkyl, C₃₋₆heterocyclyl-C(═O)—C₁₋₆alkyl, C₆₋₁₀aryl, C₆₋₁₀aryl-C(═O)—, C₃₋₁₀cycloalkyl, C₄₋₈cycloalkenyl, C₃₋₆heterocyclyl and C₃₋₆heterocyclyl-C(═O)—; wherein said C₁₋₁₀alkyl, C₂₋₁₀alkenyl, C₁₋₁₀alkoxy, C₆₋₁₀aryl-C₁₋₆alkyl, C₆₋₁₀aryl-C(═O)—C₁₋₆alkyl, C₃₋₁₀cycloalkyl-C₁₋₆alkyl, C₄₋₈cycloalkenyl-C₁₋₆alkyl, C₃₋₆heterocyclyl-C₁₋₆alkyl, C₃₋₆heterocyclyl-C(═O)—C₁₋₆alkyl, C₆₋₁₀aryl, C₆₋₁₀aryl-C(═O)—, C₃₋₁₀cycloalkyl, C₄₋₈cycloalkenyl, C₃₋₆heterocyclyl or C₃₋₆heterocyclyl-C(═O)— is optionally substituted by one or more groups selected from carboxy, —(C═O)—NH₂, halogen, cyano, nitro, methoxy, ethoxy, methyl, ethyl, hydroxy, —N(R⁶)—C(═O)R⁵, —S(═O)₂—NR⁵R⁶, —C(═O)—NR⁵R⁶, —NH—C(═O)—NR⁵R⁶ and —NR⁵R⁶;
R² is selected from the group consisting of C₁₋₁₀alkyl, C₂₋₁₀alkenyl, C₂₋₁₀alkynyl, C₃₋₈cycloalkyl, C₃₋₈cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl and C₃₋₆heterocycloalkyl, wherein said C₁₋₁₀alkyl, C₂₋₁₀alkenyl, C₂₋₁₀alkynyl, C₃₋₈cycloalkyl, C₃₋₈cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl or C₃₋₆heterocycloalkyl used in defining R² is optionally substituted by one or more groups selected from carboxy, —(C═O)—NH₂, halogen, cyano, nitro, methoxy, ethoxy, methyl, ethyl, hydroxy, and —NR⁵R⁶;
R³ is selected from C₁₋₆alkyl, C₂₋₆alkenyl, C₃₋₆cycloalkyl, C₃₋₆cycloalkyl-C₁₋₄alkyl, C₂₋₅heteroaryl, C₂₋₅heteroaryl-C₁₋₄alkyl, C₂₋₅heterocycloalkyl, C₂₋₅heterocycloalkyl-C₁₋₄alkyl, phenyl and benzyl, wherein said C₁₋₆alkyl, C₂₋₆alkenyl, C₃₋₆cycloalkyl, C₃₋₆cycloalkyl-C₁₋₄alkyl, C₂₋₅heteroaryl, C₂₋₅heteroaryl-C₁₋₄alkyl, C₂₋₅heterocycloalkyl, C₂₋₅heterocycloalkyl-C₁₋₄alkyl, phenyl or benzyl is optionally substituted by one or more groups selected from C₁₋₆alkyl, carboxy, halogen, cyano, nitro, methoxy, ethoxy, hydroxy, and —NR⁵R⁶; and
R⁴ is selected from C₁₋₆alkyl, carboxy, halogen, cyano, nitro, methoxy, ethoxy, hydroxy, and —NR⁵R⁶; 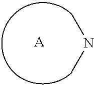
is a 4, 5 or 6-membered heterocycle which optionally contains one or two additional heteroatoms selected from O, S and N on its ring in addition to the nitrogen shown;
X is selected from —O—C(═O)—, —C(═O)—NH—, —NH—C(═O)—, —NHR⁷—C(═O)—, —C(═O)—NHCH₂—, —NH—C(═O)CH₂—, —NH—C(═O)—NH—, —O—C(═O)—NH—, —NH—(CH₂)ₘ—, —O—(CH₂)ₘ—, —C(═O)—O—, and —NH—C(═O)—O—;
wherein R⁵ and R⁶ are independently selected from —H, C₁₋₆alkyl optionally substituted with —OH, methoxy, ethoxy or halogen, C₃₋₆cycloalkyl-C₀₋ₘalkyl optionally substituted with —OH, methoxy, ethoxy or halogen, C₂₋₆alkenyl optionally substituted with —OH, methoxy, ethoxy or halogen, and a divalent C₁₋₆alkylene optionally substituted with —OH, methoxy, ethoxy or halogen that together with another divalent R⁵ or R⁶ form a portion of a ring;
R⁷ is C₁₋₆alkyl, and
m is 0, 1, 2 or 3.

2. A compound as claimed in claim 1, wherein
R¹ is selected from C₃₋₇cycloalkyl-C₁₋₂alkyl and C₂₋₆heterocycloalkyl-C₁₋₂alkyl, wherein said C₃₋₇cycloalkyl or C₂₋₆heterocycloalkyl is optionally substituted with one or more groups selected from carboxy, —C(═O)—NH₂, halogen, cyano, nitro, methoxy, ethoxy, methyl, ethyl, hydroxy, and amino.

3. A compound as claimed in claim 1, wherein
R¹ is selected from cyclohexylmethyl and tetrahydropyranylmethyl wherein said cyclohexylmethyl or tetrahydropyranylmethyl is optionally substituted with one or more groups selected from carboxy, —C(═O)—NH₂, halogen, cyano, nitro, methoxy, ethoxy, methyl, ethyl, hydroxy, and amino.

4. A compound as claimed in claim 3, wherein
R¹ is selected from cyclohexylmethyl and tetrahydropyranylmethyl wherein said cyclohexylmethyl or tetrahydropyranylmethyl is optionally substituted with one or more groups selected from methyl, hydroxy, chloro, fluoro and bromo.

5. A compound as claimed in claim 4, R¹ is selected from cyclohexylmethyl and tetrahydropyran-4-ylmethyl wherein said cyclohexylmethyl or tetrahydropyran-4-ylmethyl is optionally substituted with one or more groups selected from chloro and fluoro.

6. A compound as claimed in claim 1, R¹ is selected from cyclohexylmethyl, (4,4-difluorocyclohexyl)methyl, (4-fluorocyclohexyl)methyl and tetrahydro-2H-pyran-4-ylmethyl.

7. A compound as claimed in claim 1, wherein
R² is selected from C₁₋₆alkyl, C₂₋₆alkenyl, C₃₋₆cycloalkyl, and C₃₋₆cycloalkyl-C₁₋₂alkyl, wherein said C₁₋₆alkyl, C₂₋₆alkenyl, C₃₋₆cycloalkyl, or C₃₋₆cycloalkyl-C₁₋₂alkyl is optionally substituted by one or more groups selected from halogen, methoxy, ethoxy, methyl, ethyl, and hydroxy.

8. A compound as claimed in claim 1, wherein
R² is selected from ethyl, propyl, isopropyl, n-butyl, isobutyl, t-butyl, 1-pentyl, 2-pentyl, 3-pentyl, 1,1-dimethyl-1-propyl, 3-methyl-1-butyl, and 2,2 dimethyl-1-propyl, wherein said propyl, isopropyl, n-butyl, isobutyl, t-butyl, 1-pentyl, 2-pentyl, 3-pentyl, 1,1-dimethyl-1-propyl, 3-methyl-1-butyl, or 2,2 dimethyl-1-propyl is optionally substituted by one or more groups selected from halogen, methoxy and ethoxy.

9. A compound as claimed in claim 1, wherein
R² is selected from propyl, isopropyl, n-butyl, isobutyl, t-butyl, 1-pentyl, 2-pentyl, 3-pentyl, 1,1-dimethyl-1-propyl, 3-methyl-1-butyl, 1,1,-difluoroethyl and 2,2 dimethyl-1-propyl.

10. A compound as claimed in claim 1, wherein
R² is selected from t-butyl, 1,1,-difluoroethyl and 1,1-dimethyl-1-propyl.

11. A compound as claimed in claim 1, wherein
R³ is selected from hydrogen, C₁₋₄alkyl, halogenated C₁₋₄alkyl, hydroxy-C₁₋₄alkyl, C₃₋₆cycloalkyl, C₃₋₆cycloalkyl-C₁₋₂alkyl, methoxy-C₁₋₄alkyl, ethoxy-C₁₋₄alkyl, and C₂₋₄alkenyl.

12. A compound as claimed in claim 1, wherein
R⁴ is selected from hydrogen, hydroxy, halogen, isocyanato, methoxy, ethoxy, C₁₋₄alkyl, halogenated C₁₋₄alkyl, phenyl, benzyl, amino, C₃₋₆cycloalkyl, C₃₋₆cycloalkyl-C₁₋₂alkyl, and C₁₋₄alkoxymethyl.

13. A compound as claimed in claim 1, wherein 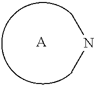
is selected from piperidinyl, isoxazolindinyl, azetidinyl, morpholinyl, pyrazolyl, pyrrolyl and pyrrolidinyl.

14. A compound as claimed in claim 1, wherein
R⁴ is hydrogen.

15. A compound as claimed in claim 1, wherein
X is selected from —O—C(═O)—, —C(═O)—NH—, —NH—C(═O)—, —C(═O)—NHCH₂—, —NH—C(═O)CH₂—, —NH—C(═O)—NH—, —O—C(═O)—NH—, —NH—, —O—, —C(═O)—O—, and —NH—C(═O)—O—.

16. A compound as claimed in claim 1, wherein —X—R³ is selected from cyclobutanylcarbonylamino, hydrocarbonyl, 2-hydroxyethylaminocarbonyl, isopropylaminocarbonyl, cyclobutanylaminocarbonyl, ethylaminocarbonyl, cyclopropylaminocarbonyl, methoxycarbonyl, ethoxycarbonyl, t-butoxycarbonyl, t-butoxycarbonylamino, allylaminocarbonyl, methylaminocarbonyl, aminocarbonyl, 2-fluoroethylaminocarbonyl, propylaminocarbonyl, cyclopropylmethylaminocarbonyl, cyclobutylmethylaminocarbonyl, t-butoxycarbonylamino, ethylaminocarbonylamino, isocyanato, cyclopropylaminocarbonylamino, 2-hydroxyethylaminocarbonylamino, ethylaminocarboxy, acetylamino, propionylamino, ethylaminocarbonylmethyl, 2-fluoroethylaminocarbonylmethyl, 2,2-difluoroethylaminocarbonyl, 2,2-difluoroethylaminocarbonylmethyl, acetylaminomethyl, cyclopropylcarbonylaminomethyl, propionylaminomethyl, and methylaminocarbonylmethyl.

17. A compound selected from:
1-({2-tert-Butyl-1-[(4,4-difluorocyclohexyl)methyl]-1H-benzimidazol-5-yl}carbonyl)-N-methylazetidine-3-carboxamide;
1-({2-tert-Butyl-1-[(4,4-difluorocyclohexyl)methyl]-1H-benzimidazol-5-yl}carbonyl)-N-cyclopropylpiperidine-3-carboxamide;
1-({2-tert-Butyl-1-[(4,4-difluorocyclohexyl)methyl]-1H-benzimidazol-5-yl}carbonyl)-N-ethylpipendine-3-carboxamide;
1-({2-tert-Butyl-1-[(4,4-difluorocyclohexyl)methyl]-1H-benzimidazol-5-yl}carbonyl)-N-cyclopropylpiperidine-4-carboxamide;
1-({2-tert-Butyl-1-[(4,4-difluorocyclohexyl)methyl]-1H-benzimidazol-5-yl}carbonyl)-N-ethylpipendine-4-carboxamide;
1-({2-tert-Butyl-1-[(4,4-difluorocyclohexyl)methyl]-1H-benzimidazol-5-yl}carbonyl)-N-methylpipendine-4-carboxamide;
N-(tert-Butyl)-1-({2-tert-butyl-1-[(4,4-difluorocyclohexyl)methyl]-1H-benzimidazol-5-yl}carbonyl)pipendine-4-carboxamide;
1-({2-tert-Butyl-1-[(4,4-difluorocyclohexyl)methyl]-1H-benzimidazol-5-yl}carbonyl)-N-cyclobutylpiperidine-4-carboxamide;
s1-({2-tert-Butyl-1-[(4,4-difluorocyclohexyl)methyl]-1H-benzimidazol-5-yl}carbonyl)-N-isoxazol-3-ylpipendine-4-carboxamide;
1-({2-tert-Butyl-1-[(4,4-difluorocyclohexyl)methyl]-1H-benzimidazol-5-yl}carbonyl)-N-1,3-thiazol-2-ylpipendine-4-carboxamide;
1-({2-tert-Butyl-1-[(4,4-difluorocyclohexyl)methyl]-1H-benzimidazol-5-yl}carbonyl)-N-(5-methylisoxazol-3-yl)pipendine-4-carboxamide;
1-({2-tert-Butyl-1-[(4,4-difluorocyclohexyl)methyl]-1H-benzimidazol-5-yl}carbonyl)-N-ethyl-N-methylpipendine-4-carboxamide;
1-({2-tert-Butyl-1-[(4,4-difluorocyclohexyl)methyl]-1H-benzimidazol-5-yl}carbonyl)-N-(cyclopropylmethyl)piperidine-4-carboxamide;
1-({2-tert-Butyl-1-[(4,4-difluorocyclohexyl)methyl]-1H-benzimidazol-5-yl}carbonyl)-N-cyclopropylpyrrolidine-3-carboxamide;
2-tert-Butyl-1-[(4,4-difluorocyclohexyl)methyl]-5-[(4-methoxypipendin-1-yl)carbonyl]-1H-benzimidazole;
2-tert-Butyl-1-[(4,4-difluorocyclohexyl)methyl]-5-[(4-ethoxypipendin-1-yl)carbonyl]-1H-benzimidazole;
2-tert-Butyl-5-{[4-(cyclopropylmethoxy)piperidin-1-yl]carbonyl}-1-[(4,4-difluorocyclohexyl)methyl]-1H-benzimidazole;
2-tert-Butyl-5-{[4-(cyclobutylmethoxy)pipendin-1-yl]carbonyl}-1-[(4,4-difluorocyclohexyl)methyl]-1H-benzimidazole;
2-tert-Butyl-1-[(4,4-difluorocyclohexyl)methyl]-5-[(4-propoxypiperidin-1-yl)carbonyl]-1H-benzimidazole;
2-tert-Butyl-1-[(4,4-difluorocyclohexyl)methyl]-5-[(4-isopropoxypiperidin-1-yl)carbonyl]-1H-benzimidazole;
2-tert-Butyl-1-[(4,4-difluorocyclohexyl)methyl]-5-{[4-(2,2-dimethylpropoxy)piperidin-1-yl]carbonyl}-1H-benzimidazole;
5-{[4-(Benzyloxy)piperidin-1-yl]carbonyl}-2-tert-butyl-1-[(4,4-difluorocyclohexyl)methyl]-1H-benzimidazole;
2-tert-Butyl-1-[(4,4-difluorocyclohexyl)methyl]-5-[(4-isobutoxypiperidin-1-yl)carbonyl]-1H-benzimidazole;
2-[1-({2-tert-Butyl-1-[(4,4-difluorocyclohexyl)methyl]-1H-benzimidazol-5-yl}carbonyl)azetidin-3-yl]-N-cyclopropylacetamide;
N-[1-({2-tert-Butyl-1-[(4,4-difluorocyclohexyl)methyl]-1H-benzimidazol-5-yl}carbonyl)piperidin-4-yl]cyclopropanecarboxamide;
1-({2-tert-Butyl-1-[(4,4-difluorocyclohexyl)methyl]-1H-benzimidazol-5-yl}carbonyl)-N-(cyclopropylmethyl)piperidin-4-amine;
4-({2-tert-Butyl-1-[(4,4-difluorocyclohexyl)methyl]-1H-benzimidazol-5-yl}carbonyl)-N-cyclopropylpiperazine-1-carboxamide;
2-tert-Butyl-5-{[4-(cyclopropylcarbonyl)piperazin-1-yl]carbonyl}-1-[(4,4-difluorocyclohexyl)methyl]-1H-benzimidazole;
2-[1-({2-tert-Butyl-1-[(4,4-difluorocyclohexyl)methyl]-1H-benzimidazol-5-yl}carbonyl)piperidin-4-yl]-N-cyclopropylacetamide;
2-tert-Butyl-5-{[3-(cyclopropylmethoxy)azetidin-1-yl]carbonyl}-1-[(4,4-difluorocyclohexyl)methyl]-1H-benzimidazole;
1-{[2-tert-Butyl-1-(cyclohexylmethyl)-1H-benzimidazol-5-yl]carbonyl}-N-cyclopropylpiperidine-4-carboxamide;
and pharmaceutically acceptable salts thereof.

18-22. (canceled)

23. A pharmaceutical composition comprising a compound according to claim 1 and a pharmaceutically acceptable carrier.

24. A method for the therapy of functional gastrointestinal disorders in a warm-blooded animal, comprising the step of administering to said animal in need of such therapy a therapeutically effective amount of a compound according to claim 1.

25. A method for the therapy of irritable bowel syndrome in a warm-blooded animal, comprising the step of administering to said animal in need of such therapy a therapeutically effective amount of a compound according to claim 1.

26. A method for preparing a compound of formula I, 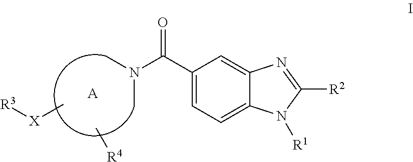
comprising the step of reacting a compound of formula II, 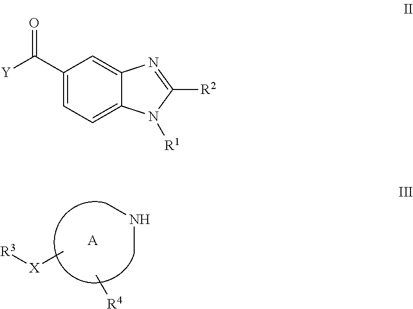
with a compound of formula III,
wherein
Y is selected from Cl, Br, F and OH;
R¹ is selected from C₁₋₁₀alkyl, C₂₋₁₀alkenyl, C₁₋₁₀alkoxy, C₆₋₁₀aryl-C₁₋₆alkyl, C₆₋₁₀aryl-C(═O)—C₁₋₆alkyl, C₃₋₁₀cycloalkyl-C₁₋₆alkyl, C₄₋₈cycloalkenyl-C₁₋₆alkyl, C₃₋₆heterocyclyl-C₁₋₆alkyl, C₃₋₆heterocyclyl-C(═O)—C₁₋₆alkyl, C₆₋₁₀aryl, C₆₋₁₀aryl-C(═O)—, C₃₋₁₀cycloalkyl, C₄₋₈cycloalkenyl, C₃₋₆heterocyclyl and C₃₋₆heterocyclyl-C(═O)—; wherein said C₁₋₁₀alkyl, C₂₋₁₀alkenyl, C₁₋₁₀alkoxy, C₆₋₁₀aryl-C₁₋₆alkyl, C₆₋₁₀aryl-C(═O)—C₁₋₆alkyl, C₃₋₁₀cycloalkyl-C₁₋₆alkyl, C₄₋₈cycloalkenyl-C₁₋₆alkyl, C₃₋₆heterocyclyl-C₁₋₆alkyl, C₃₋₆heterocyclyl-C(═O)—C₁₋₆alkyl, C₆₋₁₀aryl, C₆₋₁₀aryl-C(═O)—, C₃₋₁₀cycloalkyl, C₄₋₈cycloalkenyl, C₃₋₆heterocyclyl or C₃₋₆heterocyclyl-C(═O)— is optionally substituted by one or more groups selected from carboxy, —(C═O)—NH₂, halogen, cyano, nitro, methoxy, ethoxy, methyl, ethyl, hydroxy, —N(R⁶)—C(═O)R⁵, —S(═O)₂—NR⁵R⁶, —C(═O)—NR⁵R⁶, —NH—C(═O)—NR⁵R⁶ and —NR⁵R⁶;
R² is selected from the group consisting of C₁₋₁₀alkyl, C₂₋₁₀alkenyl, C₂₋₁₀alkynyl, C₃₋₈cycloalkyl, C₃₋₈cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl and C₃₋₆heterocycloalkyl, wherein said C₁₋₁₀alkyl, C₂₋₁₀alkenyl, C₂₋₁₀alkynyl, C₃₋₈cycloalkyl, C₃₋₈cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl or C₃₋₆heterocycloalkyl used in defining R² is optionally substituted by one or more groups selected from carboxy, —(C═O)—NH₂, halogen, cyano, nitro, methoxy, ethoxy, methyl, ethyl, hydroxy, and —NR⁵R⁶;
R³ is selected from C₁₋₆alkyl, C₂₋₆alkenyl, C₃₋₆cycloalkyl, C₃₋₆cycloalkyl-C₁₋₄alkyl, C₂₋₅heteroaryl, C₂₋₅heteroaryl-C₁₋₄alkyl, C₂₋₅heterocycloalkyl, C₂₋₅heterocycloalkyl-C₁₋₄alkyl, phenyl and benzyl, wherein said C₁₋₆alkyl, C₂₋₆alkenyl, C₃₋₆cycloalkyl, C₃₋₆cycloalkyl-C₁₋₄alkyl, C₂₋₅heteroaryl, C₂₋₅heteroaryl-C₁₋₄alkyl, C₂₋₅heterocycloalkyl, C₂₋₅heterocycloalkyl-C₁₋₄alkyl, phenyl or benzyl is optionally substituted by one or more groups selected from C₁₋₆alkyl, carboxy, halogen, cyano, nitro, methoxy, ethoxy, hydroxy, and —NR⁵R⁶; and
R⁴ is selected from C₁₋₆alkyl, carboxy, halogen, cyano, nitro, methoxy, ethoxy, hydroxy, and —NR⁵R⁶; 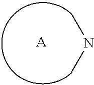
is a 4, 5 or 6-membered heterocycle which optionally contains one or two additional heteroatoms selected from O, S and N on its ring in addition to the nitrogen shown;
X is selected from —O—C(═O)—, —C(═O)—NH—, —NH—C(═O)—, —NHR⁷—C(═O)—, —C(═O)—NHCH₂—, —NH—C(═O)CH₂—, —NH—C(═O)—NH—, —O—C(═O)—NH—, —O—(CH₂)ₘ—, —C(═O)—O—, and —NH—C(═O)—O—;
wherein R⁵ and R⁶ are independently selected from —H, C₁₋₆alkyl optionally substituted with —OH, methoxy, ethoxy or halogen, C₃₋₆cycloalkyl-C₀₋ₘalkyl optionally substituted with —OH, methoxy, ethoxy or halogen, C₂₋₆alkenyl optionally substituted with —OH, methoxy, ethoxy or halogen, and a divalent C₁₋₆alkylene optionally substituted with —OH, methoxy, ethoxy or halogen that together with another divalent R⁵ or R⁶ form a portion of a ring;
R⁷ is C₁₋₆alkyl, and
m is 0, 1, 2 or 3.
